(19) United States
(12) Patent Application Publication
(10) Pub. No.: US 2022/0401587 A1
(43) Pub. Date: Dec. 22, 2022

(54) LOW X-RAY ATTENUATION CHANGE HARD SHELLED ORAL CONTRAST MATERIAL

(71) Applicant: THE REGENTS OF THE UNIVERSITY OF CALIFORNIA, Oakland, CA (US)

(72) Inventors: Benjamin M. YEH, Hillsborough, CA (US); Yuxin Sun, San Francisco, CA (US)

(21) Appl. No.: 17/736,957

(22) Filed: May 4, 2022

Related U.S. Application Data
(60) Provisional application No. 63/184,708, filed on May 5, 2021.

(51) Int. Cl. A61K 49/04

(52) U.S. Cl. CPC A61K 49/04

(57) ABSTRACT

The present invention provides a hollow borosilicate microparticle contrast media for use in CT imaging with shell material containing less than 8% oxides of non-silicon elements with atomic number greater than 10. In an exemplary embodiment, the invention provides an enteric contrast medium formulation which provides CT numbers distinct from those of water, soft tissue, and fat. In an exemplary embodiment, the invention provides an enteric contrast medium formulation that shows iodine concentrations less than 1.0 mg iodine/mL at dual energy CT or multi-energy CT image reformations. An exemplary formulation comprises, (a) an enteric contrast medium comprising a hollow borosilicate microparticle suspended in water. Exemplary hollow borosilicate microparticle has a true gravity between 0.1 and 0.4 g/cm³. In various embodiments, the hollow borosilicate microparticle is suspended in aqueous media by an agent compatible with enteric administration of the formulation to a subject in need of such administration. In an exemplary embodiment, the contrast material is incorporated into a pharmaceutically acceptable carrier in which the material is suspended. In an exemplary embodiment, the hollow borosilicate microparticle comprises 1% to 15% of the weight of an aqueous contrast material formulation. The invention also provides methods for imaging of the abdomen and pelvis by CT imaging contemporaneously with the delivery of the hollow borosilicate microparticle contrast material into the bowel lumen.

### CROSS REFERENCE TO RELATED APPLICATIONS

The present disclosure claims priority to U.S. Provisional Patent Application No. 63/184,708 filed May 5, 2021, which is hereby incorporated by reference.

### BACKGROUND OF THE INVENTION

Medical computed tomography (CT) imaging is used for the evaluation of a wide range of clinical indications, including abdominal pain, evaluation for possible malignancy, staging and monitoring of tumors, assessment of bowel injury or inflammation, and to further evaluate bowel and non-bowel disease. At CT imaging, the X-ray attenuation (CT number) of the imaged tissues is measured in Hounsfield Units (HU), which range from −1000 HU (air or vacuum, which show negligible X-ray attenuation) to 0 HU (water, which is by definition 0 HU), to over 3000 (very dense material including metal with very high X-ray attenuation).

At CT imaging, different X-ray spectra may be used to image an object such as a patient. Current CT scanners may use X-ray spectra generated from X-ray tube potentials that can be set between 70 kVp to 150 kVp, depending on the clinical requirements. Lower X-ray tube potential settings generate X-ray spectra with relatively lower energy than higher tube potential settings. For any given X-ray tube potential, the CT number of water is defined to be 0 HU, and that of air/vacuum is defined to be −1000 HU. Real life measurements in humans commonly vary by about 20 HU or so due to image noise and other artifacts. The X-ray attenuation of other materials are therefore measured relative to that of water.

Human non-fatty soft tissues such as muscle, solid organ parenchyma, and blood, which are composed mostly of atoms with atomic numbers less than 20, resemble water in so far as their CT number does not change much at low versus high X-ray tube potentials (kVp). Human non-fatty soft tissues generally show CT numbers between 20 and 70 HU regardless of tube potential setting. Similarly, room air gas molecules, which are generally composed of small atomic number molecules, tend to show CT numbers of about −1000 HU regardless of CT tube potential settings.

To better delineate anatomy, positive contrast agents are commonly delivered intravenously or orally at CT imaging. All clinical intravenous contrast agents and most positive oral contrast agents are currently based on iodine (z=53) which attenuates X-rays much more strongly than does soft tissue or water, particularly when imaged at lower kVp. Some positive oral contrast agents are based on barium (z=56) which has very similar X-ray attenuation characteristics as iodine due to their similar atomic numbers, such that iodine and barium signal are not distinguishable at CT imaging regardless of imaging technique, including at multi-energy CT.

Given that positive contrast media is generally in aqueous solution or suspension, these positive contrast agents generally show CT numbers >0 regardless of aqueous dilution. The concentration of the positive contrast material increases the CT number of the fluid or tissues in which they reside at the time of CT imaging. Typically, 1 mg iodine/mL is approximately the limit of detection of iodine in contrast-enhanced tissue and this threshold generally refers to about 20 to 25 HU of enhancement when imaged at 120 kVp, which is the most common CT kVp setting. To be visually compelling, positive contrast material generally needs to increase CT numbers on a post-contrast CT image by 50 HU or more compared to the pre-contrast CT image, corresponding to about 2 mg iodine/mL or more. Quantitative detection of contrast enhancement less than 1 mg iodine/mL is generally unreliable at clinical CT due to image noise or artifacts, though some sources believe detection of lower thresholds such as 0.8 mg iodine/mL might be reliable. Similarly, visual detection of changes of 20 HU or less from pre- to post-contrast clinical CT is considered to be unreliable.

Some oral contrast agents at CT are termed “neutral” or “negative” and closely resemble the CT number of water or soft tissue at CT (0 to 50 HU). These agents include water or solutions of water with excipients to prevent rapid resorption of water by the bowel. These oral contrast agents allow for visualization of bowel wall capillary enhancement by positive intravenous contrast agents, which is difficult to delineate when positive oral contrast agent is given. However, since these neutral contrast agents resemble the CT number of water and unenhanced soft tissue at all kVps used for medical CT imaging, these agents may make it more difficult to delineate bowel from adjacent soft tissue CT attenuation lesions such as abscesses, fluid collections, hematomas, and hypovascular or necrotic tumors which predominantly also show CT numbers of 0 to 50 HU, or marginally higher, at CT imaging across the range of CT kVps used for medical imaging.

The X-ray attenuation of CT contrast agents is traditionally determined entirely based on the concentration of the reporter atom/material in aqueous vehicle. Iodine contrast agents are described based on mg of iodine/mL. Barium sulfate contrast agents are described as w/w % barium sulfate. Neutral contrast agents have no materials that substantially change the CT attenuation from that of water (hence, 0±20 HU, which is the CT attenuation of water)

Since humans can only perceive about 30 distinct shades of gray¹ but medical CT numbers range from −1000 to over 3000 HU, medical CT images are viewed using a narrowed CT number window and level to assess different structures (bone, lung, soft tissues, etc). For soft tissue evaluation, the “level” (mid-level gray) is set at the expected CT number of soft tissue (40 or 50 HU) and the “window” is set to capture the typical range of fat and modest-to-bright positive contrast enhancement (−100 and 200 HU, respectively). To evaluate critical soft tissues, the most common window/level settings for displaying abdominal CT viewing are 350/50 HU (which displays voxels below −125 HU as pure black, and above 225 HU as pure white, and voxels between −125 and 225 HU as increasingly bright signal) or 400/40 HU (which displays voxels below −160 HU as pure black and above 240 HU as pure white, and voxels between −160 and 240 HU as increasingly bright signal).

A major problem with the use of positive and neutral oral contrast agents is non-uniform appearance of the bowel lumen contents, regardless of the viewing window/level settings. Bowel lumen commonly varies from segment to segment, with some segments containing gas, some containing fluid, some containing solids, and some containing admixtures of these materials. The enteric gas has dark CT number (−50 to −1000 HU, depending on the amount of admixed material). As such, regardless of whether positive (100 to 400 HU) or neutral oral contrast (0 to 40 HU) is used, there is almost always heterogeneity of the bowel lumen due to areas of markedly dark gas signal mixed with the oral contrast. This heterogeneity causes complexity with human or machine image interpretation.

Prior description of dark oral contrast has included insufflated gas such as room air or carbon dioxide, which creates extremely dark CT numbers close to −1000 HU in the lumen, and the insufflated gas is physically uncomfortable for the patient. These agents generally require enteric intubation and insufflation of the bowel, which is invasive. Alternatively, orally consumed contrast materials in the bowel may emit gas such as carbon dioxide via a chemical reaction. These gas-emitting agents may also cause discomfort from the gas distension or the chemicals involved. Other proposed dark oral contrast agents include perfluorocarbons that may or may not expand in volume within the bowel, and which may cause very dark signal in the bowel lumen², but which were associated with anal leakage and abdominal discomfort.

Other descriptions of dark enteric contrast include foam liquids, such as for rectal administration³ or for oral administration⁴ (US 20200000942) which can be formulated to give a dark CT number (−100 to −800 HU). These agents do not have hard shells around the gas bubbles and hence may have stability issues that preclude their use for small bowel³, or may have inhomogeneous CT numbers that range across hundreds of HU⁴. A further limitation is that these formulations may require special machines for on-site preparation⁴.

Other described dark contrast agents include oil emulsions such as Calogen⁵ or corn oil emulsions⁶⁻⁸ and paraffin suspensions⁹. These dark contrast agents may have useful mildy dark CT values of −20 to −60 HU, but result in inability to differentiate the contrast agent from natural bodily fat when imaged with conventional CT or even with dual-energy/multi-energy CT because the underlying contrasting material is made of lipids/hydrocarbons that show similar X-ray attenuation as human fat across all kVps and keVs.

Microbubble contrast agents have been described for CT, including polymer shell agents (US 005726121A, U.S. Pat. No. 5,205,290 A) which give strongly negative CT number when formulated in aqueous suspension. These contrast agents were not developed for commercial CT use. Further, the physical stability of the particles in such material is unlikely to be sufficient for use in small bowel imaging which requires at least 1 hour of stability.

Dual energy CT (DECT) and multi-energy CT (MECT) including photon-counting CT (PCCT) scanners have been developed for clinical imaging. These scanners improve on conventional single energy spectrum CT by contemporaneously evaluating the X-ray attenuation of imaged objects at different X-ray spectra. For DECT, the relative attenuation of the imaged object by of X-ray spectra of low versus high energy are compared. The low versus high X-ray energy spectra are generally obtained by setting the CT tube potential (kVp) as low (e.g. 80 kVp) or high (e.g. 140 kVp), respectively. Various other implementations are also utilized, including tin and gold filtration of the X-ray beam or use of split layer X-ray detectors to preferentially detect X-rays of lower or higher energies, or photon counting detectors to better quantify and classify the energies of the detected X-rays.

These DECT, MECT, and PCCT scanners exploit the fact that atoms in the imaged object will attenuate X-rays of different energies to characteristic degrees, based on atomic number. Since water is assigned a CT number of 0 HU and vacuum/room air is assigned a CT number of −1000 HU regardless of the X-ray energy used to image the water or air, the CT number of all other materials is determined relative to these two standards.

The z-effective of H₂O is about 7.5 which is the weighted average of the component atoms oxygen (z=8) and hydrogen (z=1). Owing to the small size and effect of protons on the overall X-ray attenuation of water, the z-effective of water is much closer to that of oxygen. Since the CT number of water is by definition 0 HU regardless of kVp settings, the CT number of other materials at low and high kVp settings are determined relative to that of water. Generally, molecules with z-effective smaller than water (such as fat/hydrocarbons which are made of carbon z=6 and hydrogen z=1) will show relatively lower CT numbers at low kVp than at high kVp (FIG. 2). And molecules with z-effective larger than that of water (such as iodine, z=53) will show relatively higher CT numbers at low than at high kVp (FIG. 2). Soft tissue, being composed of mostly carbon, hydrogen, oxygen, and nitrogen, has a slightly higher CT number than water of about 20 to 50 HU, but has a z-effective that is similar to water, and so does not show much higher CT number at low kVp than at high kVp CT settings.

A primary value of DECT, MECT, and PCCT, is the ability to quantify the amount of intravenous contrast material enhancement in image voxels without the need for acquiring a separate unenhanced CT scan. Iodine shows markedly higher CT number at low kVp than at high kVp CT settings. In aqueous solution, the 80:140 kVp CT number ratio of iodine is about 1.75, while that of water is 1.0 (by definition) and that of soft tissue is 1.05 or so. This difference can be used to quantify iodine at DECT into iodine maps. Similar methods are used for MECT and PCCT quantification of iodine contrast enhancement without the need for a separate noncontrast CT scan. Due to noise in CT data in living organisms, which show CT image artifacts due to many reasons, iodine is not reliably quantified below about 1 mg Iodine/mL. CT artifacts commonly arise due to quantum mottle, mass attenuation in thick body parts, bones, motion, metal, and non-circular shape of the imaged object. Typically, iodine map images are reconstructed as a pair with a water map image, also known as virtual noncontrast (VNC) or virtual unenhanced (VUE) image. The iodine map can be considered to be the opposite of the water map. The CT number values from the parent low and high kVp images are divided such that iodine values are assigned to the iodine map and the other values assigned to the water map.

Unlike conventional CT which use detectors that integrate the sum of X-ray energies that hit the detector, PCCT scanners use special X-ray detectors that determine the energy of each individual X-ray that hits the detector. Since the X-ray energy spectra produced by the X-ray source is known, the photon counting CT can therefore determine which X-rays were preferentially attenuated by the imaged object. This allows even better differentiation of imaged elements/materials than possible with dual energy CT. Collectively, dual energy CT and photon counting CT are termed multi-energy CT.

DECT, MECT, and PCCT images can be reconstructed to simulate the appearance of the CT scans at different monoenergetic X-ray energies. The iodine map and water map pairs are used to determine the voxel intensity of a simulated monoenergetic CT scan at any given keV, usually chosen between 40 keV and 200 keV. Different materials show characteristic CT number curves when plotted against keVs (FIG. 2)

### BRIEF SUMMARY OF THE INVENTION

The present invention substantially refines the formulation of regular hollow borosilicate microparticle (RHBM) oral contrast agents to allow the agents of the present invention to delineate the anatomy of small bowel in a more precise manner than previously described when small bowel was imaged at conventional CT and multi-energy CT. Achievement of a favored agent involved several unexpected innovations and fortuitous discovery of beneficial properties of high silicon hollow borosilicate microparticles (HSHBM), which differ from regular hollow borosilicate microparticles (RHBM) in surprising ways at CT imaging. Furthermore, targeting of specific CT number ranges for enteric contrast agents allows for surprising improvement in bowel anatomic detail delineation, regardless of whether or not conventional CT or DECT or multi-energy CT is used.

The shell material of most hollow borosilicate microparticles, or RHBM, includes a blend of silicon dioxide (SiO₂, generally >60%, Si z=14) and boron oxide (B₂O₃, >5%, B z=5) with minor amounts of multiple other oxides, including sodium (Na₂O, Na z=11), aluminum (Al₂O₃, Al z=13), magnesium (MgO, Mg z=12), calcium (CaO, Ca z=20), and zinc (ZnO, Zn z=30) oxides as well as other trace materials of even higher atomic number. At CT, the X-ray attenuation of conventional contrast agents is generally dominated by the most common atoms and the high atomic number atoms. Prior borosilicate glass particles showed substantially greater attenuation of X-rays when imaged at CT with lower kVp than with higher kVp. This relative attenuation is in relation to that of water (H₂O), which by definition is assigned a CT number of 0 HU regardless of the kVp used to image at CT.

US20180110492A1 further discloses adding barium (z=56) or other oxides of high atomic number to further increase the 80:140 kVp CT number ratio which further increases the calculated but artifactual iodine concentration on iodine maps at MECT.

Prior work on pure silicon dioxide as an oral CT contrast agent (US20140276021A1) showed that the 80:140 kVp CT number ratio was between 1.25 and 1.56, meaning that there was a substantial difference of 25 to 56% higher CT number for silicon dioxide when imaged at 80 kVp than at 140 kVp, and this ratio is far from 1.0.

As such, it was an unexpected and counterintuitive surprise to find that a high silicon hollow borosilicate microparticle (HSHBM) with very high amounts of silicon dioxide and very low amounts of boron and other oxides could be engineered into a contrast agent to show an 80:140 kVp CT number ratio close to 1.0, with only minimally higher X-ray attenuation at low versus high kVp CT imaging compared with RHBMs.

Based on z-effective, one would predict that the shell material of HSHBM would have a higher z-effective and hence show a greater relative CT number at low than at high kVp's compared with the shell of RHBM, but the opposite was found to be true. An exemplary high silicon borosilicate has 92% or more oxide of silicon (z=14) with <2% boron (z=4) trioxide (which has a relatively small z-effective). By comparison, regular borosilicate has only about 70% to 80% oxide of silicon, a large amount of boron trioxide (about 15%), with most of the rest of the composition due to oxides of Na, Mg, and Al (z=11, 12, and 13, respectively, atoms of which are each smaller than silicon z=14).

Only on further evaluation is it clearer that the oxide of silicon has only one silicon atom per two oxygen atoms of SiO₂ (molar ratio 1:2), hence the z-effective of SiO₂ is actually similar or smaller than that of the z-effective of the oxides of Na, Mg, and Al which each have a higher molar ratio relative to oxygen of 2:1, 1:1, and 2:3. Also on further analysis, high silicon borosilicate has relatively lower amounts of other small proportion materials that are seen in standard borosilicate, including oxides of calcium (z=20) and zinc (z=30) oxides as well as other atoms with higher atomic numbers.

The use of HSHBM for medical imaging purposes is novel. Generally, HSHBM is used to blend with other materials to physically lighten those materials, such as for aerospace or marine applications, or HSHBM is used for electronics and devices where low dielectric effects are needed, or HSHBM is used for thermoablative products (heat shields). One description of HSHBM for a medical device uses these materials to physically lighten the weight of breast implants by incorporating the HSHBM into silicone polymer gels (US20120277860A1). That breast implant patent description does not suggest use of the HSHBM as diagnostic medical imaging contrast material. As such, the use of HSHBM for a diagnostic medical imaging purpose is non-obvious.

A further innovation of our invention is the use of lower true density hollow borosilicate particles than previously tested for RHBM contrast agents. Although US20180110492A disclosed a range of possible specific gravity particles for use in contrast materials, it described the value of using particles with specific gravity similar to that of water (closer to 1 g/mL) because such particles may be easier to suspend in aqueous formulation. The disclosure is silent with respect to hollow borosilicate particles with specific gravity lower than 0.45 g/cm³. Aqueous formulations with concentrations of hollow borosilicate particles below 20% w/w are not expressly disclosed. Surprisingly, the present invention achieves its efficacious results using aqueous formulations with concentrations of HBMs of from about 0.5 to about 10% w/w.

In an exemplary embodiment, the invention provides a sterile aqueous pharmaceutical formulation of low-density hollow borosilicate microparticles at low concentrations. Exemplary formulations of the invention include low density hollow borosilicate microparticles at a concentration of not more than about 10%, e.g., not more than about 9%, 8%, 7%, 6%, 5%, 4%, 3%, 2%, or not more than about 1% (w/w) of the formulation. An exemplary formulation is appropriate for enteric administration to a subject immediately prior to, contemporaneous with (or a combination thereof), acquiring an image through at least a section of the subject's abdomen (e.g., bowel). The formulation is stable, wherein “stable” refers to a formulation of the invention in which a substantial portion (e.g., > about 50%, 60%, 70%, 80%, 90% or > about 95%) of the particles remain in suspension in a pharmaceutically acceptable aqueous vehicle during the period necessary to prepare and administer the formulation to the subject, and to acquire the image. Typical durations of time between oral ingestion of contrast material and image acquisition (period in which the formulation is stable) may range from a few minutes (e.g., under 20 min) for imaging of the esophagus and stomach, to from about 20 to about 120 minutes for imaging of the small bowel, to from about an hour to about 2 days for imaging of the colon.

In various embodiments, the particles are at least partially maintained in suspension by incorporation into the formulation of a suspending agent. Exemplary suspending agents are incorporated into the formulation in an amount of from about 0.1% to about 20%, e.g., from about 0.5% to about 15%, e.g., from about 1% to about 10%, e.g., from about 3% to about 8%. An exemplary formulation of the invention is prepared as a unit dosage formulation, with the dosage determined for an individual patient prior to administering the agent, and the formulation prepared in the clinical setting immediately prior to its administration to the subject.

In various embodiments, the invention provides formulations and methods using low true density hollow borosilicate microparticles at low concentrations, e.g., from about 1 to about 10% w/w, to achieve formulations with target CT number ranges of from about −20 to about −70 HU or from about −160 to about −300 HU. In other embodiments, the invention provides the use of low concentrations of low true density hollow borosilicate microparticles to achieve formulations with minimal calculated iodine concentrations of less than about 1 mg iodine/mL at dual energy CT or multi-energy CT image reconstructions. These low concentrations of low true density hollow borosilicate microparticles, in various embodiments, range from about 0.2% to about 12% w/w of the aqueous suspension. Alternatively, these low concentrations of low true density hollow borosilicate microparticles, in various embodiments, range from about 0.5% to about 9% w/w of the aqueous suspension, or from about 0.5% to about 4%, or from about 5% to about 9% of the aqueous suspension. The low true density hollow borosilicate microparticles of the invention, in various embodiments, range from a true density of about 0.10 to about 0.40 g/cm³. In other embodiments, the low true density hollow borosilicate microparticles of the invention range in true density from about 0.2 to about 0.35 g/cm³.

In various embodiments, our invention describes formulations of dark HSHBM oral contrast material such that it darkens the bowel lumen to less than about −160 HU so that the CT number is just outside the typical soft tissue window level viewing settings for CT (e.g. window/level settings of 400/40 HU, which assigns visible gray scale to voxel signal between −160 and +240 HU, and pure black to voxel signal below about −160 HU, and pure white to voxel signal above about +240 HU). In various embodiments, the invention provides a method incorporating informed selection of the HSHBM formulation to allow the bowel lumen to appear much more uniform in CT number (all dark) at typical soft tissue viewing window and level settings (FIG. 9 and FIG. 14), and thereby facilitate perception and delineation of more subtle disease, including with artificial intelligence segmentation or evaluation of the resultant CT images of scans obtained with our HSHBM formulations.

In various embodiments, the invention provides formulations of dark oral contrast material such that it darkens the bowel lumen to a value from about 50 to about 300 HU below that of the CT number of bowel wall so that the thickness of the bowel wall can be more accurately measured and perceived (FIGS. 4, 15, and 16). The informed concentration of various embodiments of the invention provides a CT number of the bowel lumen contrast material that is between that of bowel wall and that of fat, or provides a CT number of the bowel lumen contrast material that is below that of fat, but not more than about 100 HU below that of the highest HU value that is rendered as pure black on typical abdominal CT window and level settings.

In various embodiments, the invention provides formulations of dark oral contrast material such that it provides greater spatial resolution of bowel folds when viewed at typical soft tissue bowel (FIG. 15)

In various embodiments, the invention provides formulations of HSHBM as oral contrast material with CT numbers less than about −20 HU and with 80:140 kVp CT number ratio from about 0.90 to about 1.00. In various embodiments, the invention provides formulations of HSHBM contrast material showing apparent iodine concentrations of less than about 1.0 mg iodine/mL at iodine image reconstructions from dual energy CT, multienergy CT, and photon counting CT scans. In various embodiments, our invention describes formulations of HSHBM contrast material that shows apparent iodine concentrations of less than about 0.8 mg iodine/mL at iodine image reconstructions (FIG. 17) from dual energy CT, multienergy CT, and photon counting CT scans.

In various embodiments, sugar alcohols, magnesium hydroxide, polyethylene glycol, cellulose, or other materials, alone or in combination, known to increase bowel transit speed, may be added to the aqueous pharmaceutical formulation.

In various embodiments, one or more of tricalcium phosphate, powdered cellulose, magnesium stearate, sodium bicarbonate, sodium ferrocyanide, potassium ferrocyanide, calcium ferrocyanide, calcium phosphate, sodium silicate, silicon dioxide, calcium silicate, magnesium trisilicate, talcum powder, sodium aluminosilicate, potassium aluminium silicate, calcium aluminosilicate, bentonite, aluminum silicate, stearic acid, polydimethylsiloxane silica, or other flow agents may be added to improve the powder dispersion in the manufacturing or powder product (i.e., the hollow particles, or the hollow particles and one or more suspending agent or other additive of use in producing the aqueous pharmaceutical formulation prior to hydration.

In various embodiments, excipients may be added to improve the extent of bowel distension. To achieve this goal, excipients such as xanthan gum, gellan gum, guar gum, polyethylene glycol, magnesium hydroxide, cellulose, silica, sugar alcohols, or other fillers may be introduced to alter the thickness (e.g., viscosity or osmolarity) of the formulation. In various embodiments, thicker formulations prevent collapse of the bowel lumen, particularly of the proximal small bowel and stomach, compared with less viscous formulations. In various embodiments, higher osmolality formulations prevent the absorption of water from the bowel lumen, and hence maintain distension of the bowel lumen compared with lower osmolarity formulations. In various embodiments, the osmolality ranges from about 90 to about 450 milliosmoles per kilogram, or from about 120 to about 180 milliosmoles per kilogram, or from about 180 to about 295 milliosmoles per kilogram. In various embodiments, the viscosity of the contrast agent ranges from about 150 to about 2000 centipoise (cP), or from 300 to about 1500 cP, or from about 600 to about 1500 cP.

The negative enteric contrast agent of the invention may be used either without or with an intravenous contrast agent for CT imaging. The negative enteric contrast agent of the invention may be formulated to produce CT numbers that improve the conspicuity of intravenous contrast material enhancement of the bowel wall and adjacent vascularized structures.

In various embodiments, the enteric contrast agent of the invention shows one or more levels of leachable Arsenic (As), Cadmium (Cd), Lead (Pb), and Mercury (Hg) that are less than 15, 5, 5, and 30 micrograms per dose, respectively, when incubated with simulated gastric juice for 4 hours. In various embodiments, the enteric contrast agent of the invention shows levels of leachable Arsenic (As), Cadmium (Cd), Lead (Pb), and Mercury (Hg) that are less than 1.5, 0.5, 0.5, and 3.0 micrograms per dose, respectively, when incubated with simulated gastric juice for 4 hours.

In various embodiments, the invention provides CT images of dark HSHBM enteric contrast agent-enhanced CT scans of the invention to be used in conjunction with software including artificial intelligence or deep learning for segmentation of the bowel at CT and image interpretation, including the delineation of bowel from non-bowel structures, delineation of the center line of the bowel, measuring bowel segment lengths, and identifying abnormally thickened bowel wall, abnormally hyper- or hypo-enhancing bowel wall, or focal lesions of or around the bowel.

In an exemplary embodiment, the invention provides HSHBM in water suspension which is an enteric contrast medium formulation. The material is formulated in a pharmaceutically acceptable aqueous vehicle in which the particles are suspended. In an exemplary embodiment, the shell material is bound covalently or by weaker intermolecular forces to polymers, organic material, or hydrogel to improve dispersion in the aqueous media. In an exemplary embodiment, the vehicle contains additives to retain fluid in the bowel lumen. In an exemplary embodiment, the aqueous vehicle contains agents to promote intestinal motility. In an exemplary embodiment, the shell material is bound covalently or by weaker intermolecular forces to polymers, organic material, or hydrogel that decreases the CT number of the overall formulation at low CT kVp compared to high CT kVp settings.

In various embodiments, the HSHBM utilized in the formulation contain less than 5% by weight non-floating particles, e.g., broken or damaged microparticles, as well as microparticles with a small internal void and dense outer shell.

Isostatic crush strength determines the percentage volume of HBM that collapses or breaks at a specified applied pressure. Breakage of HBM may result in unwanted small irregular particles in the contrast agent and may decrease the utility of the resulting formulation. In various embodiments, the invention provides use of HSHBM where less than about 10% of the volume of HSHBM utilized in formulation breaks at 500 psi of pressure. In various embodiments, less than 3% of the volume of HSHBM utilized in formulation breaks at 500 psi of pressure. The breakage of the HSHBM, for the purpose of accurate measurement of breakage, is made when the HSHBM is in isolated dry powder form, rather than in liquid formulation. By way of illustration, in an exemplary embodiment, the volume refers to the volume of the dry powder HSHBM, prior to formulation, is measured in a pycnometer.

Nonfloating particles of HBM in aqueous suspension may be undesired because nonfloating particles may include broken or damaged microparticles, as well as microparticles with a small internal void and dense outer shell. Such particles may result in undesired layering in the bowel lumen. In various embodiments of our invention, less than about 5% of the volume of HSHBM used in formulation of the invention are nonfloating. In various embodiments of the present invention, less than about 3% of the volume of HSHBM particles are nonfloating. By way of non-limiting illustration, this measurement of nonfloating volume fraction may be made by simple floatation of a known volume of HSHBM, as determined by mass of the sample divided true gravity, in water then measuring the volume of the nonfloating fraction in mL using a separation flask having a graduated cylinder at the dependent end. Alternatively, measurement of the nonfloating volume fraction may be made by isolating and drying the floating and nonfloating fractions and measuring the volumes of each by gas pycnometer.

In an exemplary embodiment, the invention provides a contrast medium formulation that may also be delivered into the digestive system and other bodily cavities that may be natural such as the vagina or bladder, or surgically created such as neobladders, or artificial medical devices such as tubes, catheters, pouches, reservoirs, or pumps.

Additional illustrative advantages, objects and embodiments of the invention are set forth in the description that follows.

The enteric contrast agents of the invention are substantially different from microbubble contrast agents used in ultrasound imaging. Microbubbles in ultrasound are usually gas microbubbles of perfluorocarbon gas or nitrogen gas surfaced-coated by flexible material such as albumin, carbohydrates, lipids, or biocompatible polymers that allow ultrasound to cause expansion and contraction of the bubbles to thereby amplify signal at ultrasound imaging. The mean size of ultrasound contrast microbubbles is usually in the 2-6 micron range, and the common concentration level is about 10 million microbubbles per mL. It is thus calculated that less than 1% of the volume of microbubble-type ultrasound contrast formulations is gas-filled or hollow, and such a small volume fraction of gas or void space does not produce sufficiently low signal to be useful at CT imaging as a negative contrast agent. Even if the bubbles were pure gas (−1000 HU, which is the lowest HU CT number on the CT number scale), a 1% volume of microbubble in water (0 HU at CT) suspension would give a CT number of about −10 HU, which is not much different than that of water itself. Two recent review articles on ultrasound microbubble contrast agents are given here:
1) Ultrasound microbubble contrast agents: Fundamentals and application to gene and drug delivery By: Ferrara, Katherine; Pollard, Rachel; Borden, Mark. Book Series: ANNUAL REVIEW OF BIOMEDICAL ENGINEERING Volume: 9 Pages: 415-447 Published: 2007;
2) Microbubbles in medical imaging: current applications and future directions. By: Lindner, JR. NATURE REVIEWS DRUG DISCOVERY, Volume: 3 Issue: 6 Pages: 527-532 Published: June 2004

The enteric CT contrast materials of the present invention are substantially different than previous perfluorocarbon oral contrast materials proposed for CT and MR and X-ray imaging. These previous agents include liquid perfluorocarbon, which may or may not be emulsified; the perfluorocarbon may or may not be brominated. In these previous agents, the perfluorocarbon may expand into a gas at body temperature and create negative contrast signal and further bowel distension. Drawbacks of perfluorocarbon agents are that they may be difficult to administer, have an oily texture that may be unacceptable to patients, and their expansile characteristic carries safety concerns when administered into diseased bowel segments² (also U.S. Pat. Nos. 5,205,290; 4,951,673). Brominated perfluorocarbons have been described as CT contrast agents and may produce positive CT number signal.

Other embodiments, objects and advantages of the invention will be apparent from the detailed description that follows.

### BRIEF DESCRIPTION OF THE DRAWINGS

FIG. 1 shows the range of CT numbers of soft tissues, fat, neutral oral contrast (OC) and positive OC typically seen in unenhanced CT and intravenous positive contrast enhanced CT. Gaps are seen in the range between water and fat (−20 to −70 HU) and below that of fat (−120 HU). Also shown on the right of the figure is a bracket that shows the range of CT numbers displayed on typical soft tissue window/level viewing settings of 400/40 HU. Voxels between −1000 and −160 HU are displayed as black, then progressively whiter shade of gray until 240 HU, above which the voxels are displayed as pure white. Exemplary embodiments of the invention include HSHBM formulations that provide CT numbers between those of fat and water (−70 HU to −20 HU) to allow differentiation from fat and water. Exemplary embodiments of the invention include HSHBM formulations that show CT numbers below that of fat, and below the lower limit of soft tissue viewing window/level settings (−160 HU and −300 HU) to provide near uniform dark appearance of the bowel lumen when viewed at typical abdominal window and level settings.

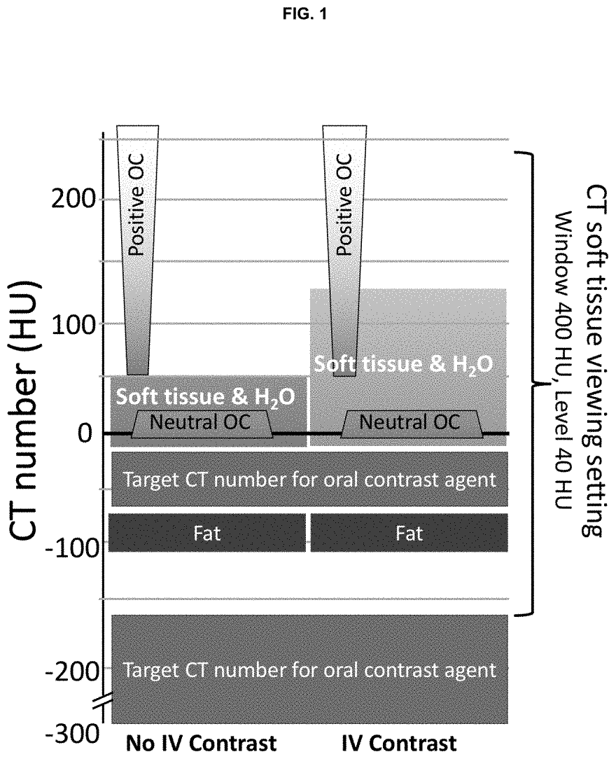

FIG. 2 is a graph of CT number of various materials (y-axis) and virtual monoenergetic image keV (x-axis). Iodine solutions show characteristically higher CT number at low keV, with diminishing CT number at higher keVs. Conversely, water remains unchanged at 0 HU. Soft tissues, such as muscle or other solid organ parenchyma, remain with nearly unchanged CT numbers across keV's, except for a minor elevation of CT number at low keVs. Aqueous suspensions of standard hollow borosilicate microparticles show negative CT numbers at high keVs, and increasing CT numbers at low keVs. The slope of the curve for these aqueous suspensions of regular hollow borosilicate microparticles (RHBM) resembles those of iodine solutions, and so such suspensions appear to show substantial unwanted apparent iodine signal at iodine maps. Unlike iodine, fat shows an upward slope in CT number with increasing keV. Various embodiments of the invention utilize aqueous suspensions of high silicon hollow borosilicate microparticles (HSHBM) which show minimal elevation of CT number at low keV image reconstructions. This minimal negative slope allows these various embodiments of the invention to be readily differentiated from fat, and the smallness magnitude of the slope prevents the various embodiments of the invention from appearing as false iodine concentrations on iodine maps. keV=kiloelectron volt.

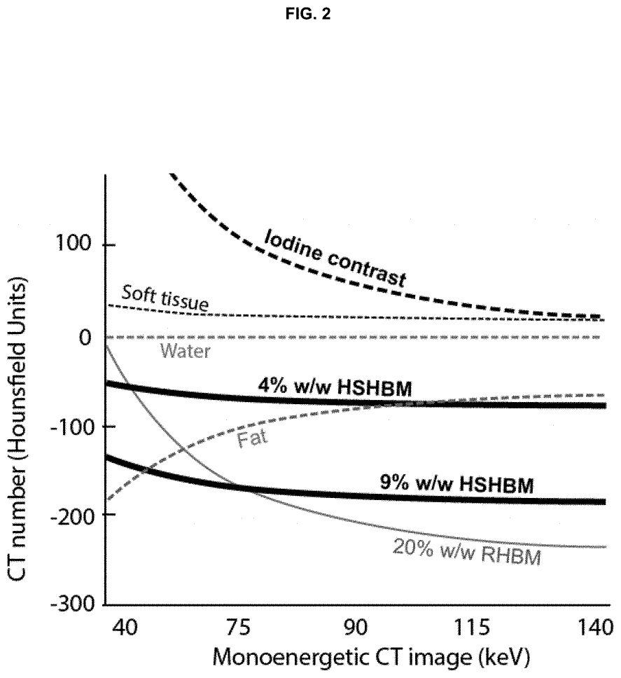

FIG. 3A-FIG. 3B shows a CT obtained with aqueous high silicon hollow borosilicate microparticles (HSHBM) enteric CT contrast material formulation. A) CT scan displayed with soft tissue window/level of 400/40 HU. B) The same CT scan image displayed with lung window/level of 1500/−600 HU. The CT number of the HSHBM CT contrast material is −200 HU in the posterior bowel (thin arrows), and is seen as intermediate gray that is darker than the surrounding fat (−100 HU) on the right CT image. The bowel wall of HSHBM enteric CT contrast material is clearly visible on the right soft tissue window/level CT image (thin arrows) as being slightly darker CT number than surrounding fat. Conversely, for the bowel that contains air (thick arrows), the bowel wall is not visible. The loss of visualization of the bowel wall for bowel filled with air is due to volume averaging of the soft tissue bowel wall (CT number 50 HU) with adjacent air (CT number −1000 HU), which renders the interface voxels to show CT numbers below −160 HU (outside the soft tissue window/level), and hence the bowel wall is rendered as black on the CT scan when viewed with standard soft tissue viewing windows.

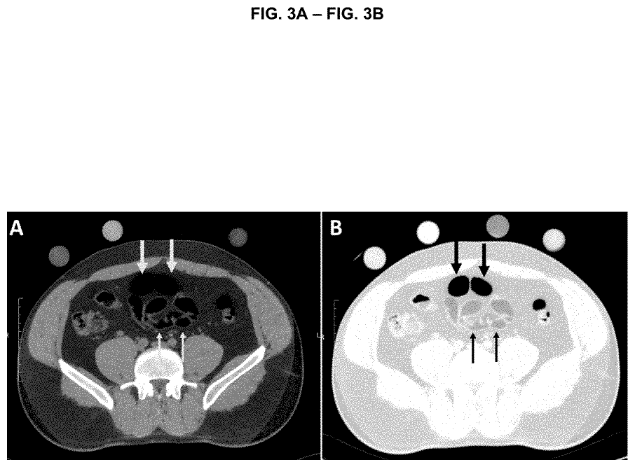

FIG. 4 shows a CT scan of different materials on a 2 mm thick plastic sheet that simulates the CT number and thickness of bowel wall to demonstrate accuracy of measurement of bowel wall with different oral contrast agents in vitro. Open-ended plastic cylinders of were attached to a diagonally placed plastic sheet that was specifically engineered to simulate the CT number of unenhanced bowel wall (CIRS phantom). The cylinders were filled with Readi-Cat 2™ barium sulfate positive oral contrast (CT number 375 HU), room air (CT number −1000 HU), VoLumen™ neutral oral contrast (CT number 23 HU), 9% w/w test article HSHBM enteric CT contrast (9% w/w HSHBM of true density 0.29 in aqueous suspension, CT number −185 HU), and 4% w/w test article HSHBM enteric CT contrast (4% w/w HSHBM of true density 0.29 in aqueous suspension, CT number −85 HU), and the set up was partially submerged in canola oil to simulate surrounding mesenteric fat CT number. The image was viewed at standard soft tissue window/level. The thickness of the bowel wall was measured using ImageJ by count of voxels within zero +/−3 standard deviations of background noise across regions of interest of 2 cm length. The measured thickness of the simulated plastic sheet bowel wall was 1.4 mm for the barium sulfate positive oral contrast, 0.9 mm for the air, 6 mm for the VoLumen neutral oral contrast, 2.0 for the 9% w/w test article HSHBM enteric CT contrast, and 2.0 for the 4% w/w test article HSHBM enteric CT contrast. These results show that exemplary HSHB aqueous suspensions provide highly accurate delineation of bowel wall in vitro compared to positive, neutral and gas CT contrast agents.

FIG. 5A-FIG. 5B shows CT abdomen image of a volunteer who consumed HSHBM with excessive concentration of HSHBM that created unwanted false iodine signal. A) After consuming 1200 mL of an aqueous suspension of 15% w/w test article HSHBM enteric CT contrast (15% w/w HSHBM of true density 0.35 in aqueous suspension, CT number −195 HU), vials containing water, the same HSHBM contrast material (small white arrow), and iodine contrast (thick white arrow) was placed on the abdomen and the patient scanned with a dual energy CT scanner. B) On iodine map overlay of the same scan, distinct iodine signal (red color) was seen in the iodine contrast vial, as well as unwanted false iodine signal in the HSHB contrast vial. Similarly, unwanted false iodine signal was seen in the bowel that contained the HSHB contrast material (small black arrows).

FIG. 6A-FIG. 6K. CT images of vials of contrast material displayed with different window and level viewing settings (top two rows) and iodine map (bottom row). Vials with low amounts of iodine (columns A and B) are shown, with column B containing iodine at 1mg iodine/mL, which is the threshold of iodine where iodine is able to be detected at dual energy CT. For comparison, a water vial containing no iodine is shown in column C. Note: this in vitro scan has much less noise than would a clinical CT scan which commonly has much more noise and artifact. Vials of 0.27 g/cm³ HSHBM aqueous suspensions are shown in columns D, E, and F. Of these, the vials with 9, 5, and 3% w/w HSHBM showed apparent iodine concentrations less than 1 mg iodine/mL, while the higher concentration HSHBM vials showed unwanted apparent iodine concentrations greater than 1 mg iodin/mL. Comparison RHBM aqueous suspensions showed high unwanted apparent iodine concentrations (columns I, J, and K). Numerical results of this CT experiment are shown in FIG. 7.

FIG. 7. CT experiments showing CT numbers, 80:140 kVp CT number ratios, and apparent iodine concentrations of various contrast agents. The HSHBM agents are 270TA and 350TA. The RHBM agents are 45P25, 60P18, iM30K, and 34P. Target CT numbers for oral contrast agent of between −20 and −70 HU were achieved by 350TA and 270TA, respectively, at 3% w/w in aqueous suspension. Target CT numbers for oral contrast agent of between −160 HU and −290 HU was achieved by use of 270TA at 9% w/w in aqueous suspension. Vials A through K correspond to CT images of FIG. 6. n.a=not applicable. Y=yes. N=no.

FIG. 8A-FIG. 8B. Demonstration of CT postprocessing display of intravenous iodinated and enteric HSHBM contrast materials without use of dual energy CT. Intravenous positive contrast enhanced CT obtained in patient after consumption of 1200 mL of 9% w/w test article HSHB enteric CT contrast (9% w/w HSHBM of true density 0.29 in aqueous suspension, CT number −180 HU). A) Volume rendered image shows the blood vessels and some organs. The enteric lumen is not displayed as opaque due to its radiolucency. B) Same image now also with volume rendered enteric contrast based on seed growing segmentation of the bowel. This type of display of both the intravenous and enteric contrast material is impossible with current neutral or positive enteric contrast agents.

FIG. 9A-FIG. 9E. Demonstration of CT postprocessing display of intravenous iodinated and enteric HSHBM contrast materials with use of dual energy CT. A) CT of abdomen after consumption of 1200 mL of 9% w/w test article HSHBM enteric CT contrast (9% w/w HSHBM of true density 0.29 in aqueous suspension, CT number −180 HU), and without intravenous contrast, displayed with soft tissue window/level viewing settings. B) subsequent dual energy CT scan with intravenous iodinated positive contrast enhanced CT obtained 2 minutes later, displayed with soft tissue window/level viewing settings. Arrows show excellent detail of stomach and jejunal anatomy with clear intravenous enhancement of the bowel walls. The bowel lumen is black, as expected, at this soft tissue window/level viewing setting, regardless of whether it contains the dark contrast agent (black arrow) or gas (white arrows), thereby providing a uniform appearance of the bowel lumen. C) iodine map overlay shows no unwanted false iodine signal in the bowel lumen. Some unwanted false iodine signal is seen in muscle, as is typical for all DECT scans, regardless of the type of oral contrast agent. D) Lung window/level viewing setting of 1500/−600 HU reveals the bowel lumen of the stomach and jejunum contains the dark contrast agent, which appears as intermediate gray that is slightly darker than that of fat CT number (arrows). E) iodine map shows no unwanted false iodine signal in the bowel lumen. Note: Some stool is present in the colon because the HSHBM contrast agent has not yet reached those segments of colon. This stool material appears as intermediate signal on the soft tissue viewing settings and as false iodine signal.

FIG. 10A-FIG. 10C. Demonstration of CT postprocessing display of intravenous iodinated and enteric HSHBM contrast materials with use of dual energy CT. A) CT of abdomen after consumption of 1200 mL of 9% w/w test article HSHBM enteric CT contrast (9% w/w HSHBM of true density 0.29 in aqueous suspension, CT number −180 HU), and with intravenous iodinated positive contrast displayed with soft tissue window/level viewing settings. The bowel lumen is dark. B) Iodine map, with iodine signal shown as orange. No unwanted false iodine signal is seen in the bowel lumen (though some unwanted false iodine signal is present in the muscle, as is common at clinical dual energy CT). C) Dual energy CT reconstruction with bowel lumen HSHBM contrast rendering as purple overlay and intravenous iodine contrast rendering as orange overlay on CT. This type of software delineation of oral from intravenous contrast from soft tissues is not readily achievable with conventional oral contrast agents because positive oral contrast resembles positive IV contrast in CT numbers, and neutral oral contrast resembles soft tissue and biological fluid in CT number.

FIG. 11. Table of CT results with various hollow microparticle aqueous suspensions. The HSHBM agents achieve less than −160 HU at CT which allows for delineation from fat at CT. Further, the HSHBM agent achieves 80:140 kVp CT number ratios above 0.90 and below 1.0, which is ideal to delineate from both fat and true iodine signal at dual energy CT. Note: the polymer phenolic hollow microspheres are also able to achieve these ranges, but are potentially toxic, less stable, and are more difficult to solubilize in aqueous suspension.

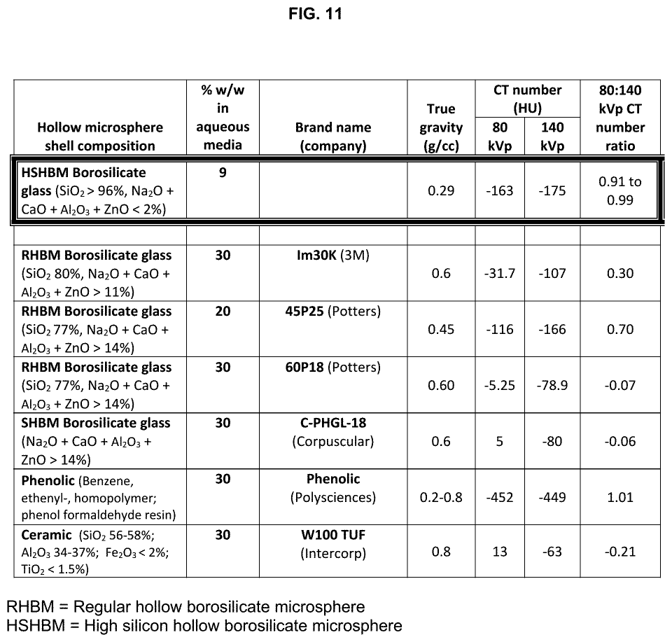

FIG. 12. Table of leachable heavy metals from various hollow borosilicate microparticles (HBM). The oral permissible daily exposures (PDE) of class 1 elemental impurities Arsenic (As), Cadmium (Cd), Lead (Pb), and Mercury (Hg) are shown in row 2 of the table, and is extrapolated to a dose of 145 grams HBM and shown in row 3 of the table. The toxicity of an elemental impurity is related to its extent of exposure. Levels of leachable elemental impurities in hollow borosilicate microparticles was evaluated via two methods. The first method used a 75% aqua regia digestion of the sample, followed by ICP-MS analysis. Results are reported in parts per million (ppm) in the table. The second method used sample incubation in a simulated gastric fluid solution for 8 hours at 40 degrees celsius to mimic an acid environment similar to the stomach. After filtration, the filtrate is analyzed via ICP-MS. Results are reported as micrograms (ug) in the table below for the Regular HBM (RHBM) and representative High-Silicon HBM (HSHBM) materials and oral aqueous formulations.

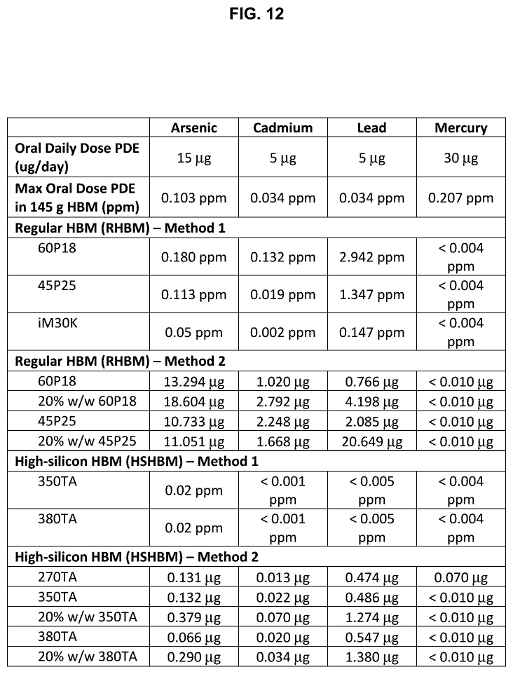

FIG. 13. Table showing comparison of oxide shell compositions of RHBM (regular hollow borosilicate microparticles) and HSHBM (high-silicon hollow borosilicate microparticles), as determined by XRF (x-ray fluorescence).

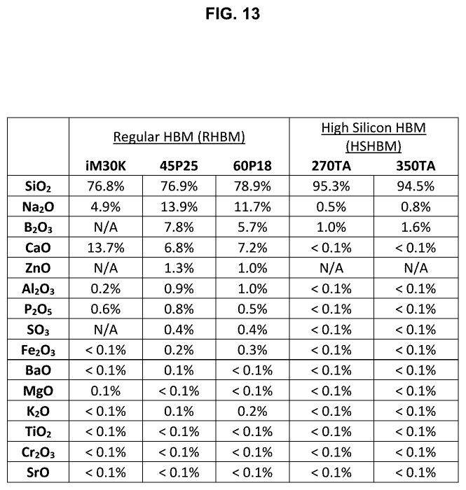

FIG. 14. Coronal CT scan of the abdomen obtained with positive oral contrast (left) and HSHBM oral contrast (right image), viewed using standard soft tissue window/level settings of 400/40 HU. The bowel lumen in the positive oral contrast scan (left) ranges in CT number from −1000 (gas) to +350, and spans the entire grayscale from black to pure white, and so it may be confusing to differentiate bowel from other structures due to the highly variable shades of gray of the bowel. The bowel lumen of the dark HSHBM oral contrast scan is much easier to delineate because it is uniformly black or near-black because the HSHBM was formulated to be −180 HU, which is just below CT number that would be displayed as black at standard soft tissue window level viewing settings. Furthermore, the blood vessels for both scans are enhanced with positive intravascular contrast, which is >200 HU, and may be difficult to distinguish from positive oral contrast-enhanced bowel (left image) but is very easy to distinguish from HSHBM oral contrast-enhanced bowel (right image). This illustrates how precisely formulated HSHBM contrast agents of our invention simplifies image interpretation, both for human readers and artificial intelligence.

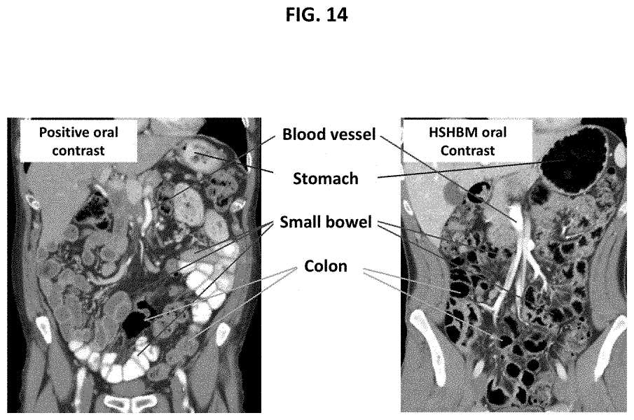

FIG. 15. Spatial resolution CT phantom scanned at 120 kVp at CT. The spatial resolution CT phantom is a plastic block with groups of 4 plastic bars between 5 identically thick hollow slits. The hollow slits and plastic columns are 1.7, 1.3, 1.0, 0.90, 0.73, and 0.57 mm thick (noted on x-axis). The plastic is 150 HU. The plastic bars simulate bowel folds at CT, which may be less than 3 mm in thickness, and down to 1 mm thick or thinner. The slits are filled with different types of oral contrast of air (−1000 HU), HSHBGM 9% w/w (−180HU), water (0 HU), and diluted iohexol 9% I/mL (220 HU). The CT images are displayed using typical abdominal window/level settings of 400/40 HU. The best spatial resolution was seen when the phantom slits were filled with HSHBGM. When filled with HSHBGM, all four plastic bars of a group could be seen down to 0.90 mm thickness/spacing, while filling with water only allowed all four bars to be seen down to 1.0 mm, with iohexol positive oral contrast down to 1.3 mm, and with air down to 1.7 mm. The large difference in HU between the HSHBGM and the plastic bars, without extending too far past the grayscale of typical abdominal window/level settings, allows the HSHBGM to best delineate the plastic bars.

FIG. 16A-FIG. 16B. Measured thickness of “bowel wall” CT phantom shows that bowel lumen contents measuring between −50 and −300 HU provides the most accurate determination of bowel wall thickness on different CT scanners and different field of views. Phantom similar to that of FIG. 4 was partly submerged in lard, then filled with different contrast agents or materials that including air (−1000 HU), various aqueous suspensions of HSHBGM to achieve HU values between −700 to −50 HU, canola oil (−110 HU), water (0 HU), saline (5 HU), dilutions of iohexol in water to achieve HU values between 50 to 600 HU, and commercial oral contrast agents Breeza (0 HU), VoLumen™ (20 HU), Omnipaque 12% I/mL (300 HU) or Readi-Cat 2™ (400 HU) (see x-axis). The 2 mm thick plastic plate was engineered to simulate unenhanced bowel wall CT attenuation of 40 HU (FIG. 16A) or 5 mg I/mL contrast-enhanced bowel wall of 165 HU (FIG. 16B). The phantom was scanned on two different CT scanners (Philips IQon and General Electric Revolution 256) at two different field of views (22 cm which gives voxel sizes of 0.43 mm², and 50 cm which gives voxel sizes of 0.98 mm²). For each scanner and field of view, the images were analyzed to measure the apparent thickness of the plastic plate by placement of a rectangular region of interest on the plastic plate and counting all voxels within 3 standard deviations of the measured HU value of the plastic plate then multiplying by the voxel area. For the unenhanced bowel wall phantom (FIG. 16A), the bowel wall thickness measurement best approximated the true 2 mm thickness for bowel lumen contrast between −50 and −300 HU. Bowel lumen contents lower than −300 HU and higher than 100 HU resulted in excessive under-estimation of bowel wall thickness, while bowel lumen contents between 0 and 100 HU resulted in excessive over-estimation of bowel wall thickness, the latter because the bowel wall CT attenuation was too close to that of the bowel lumen CT attenuation. For the IV contrast enhanced bowel wall phantom (FIG. 16B), the bowel wall thickness measurement again best approximated the true 2 mm thickness for bowel lumen contrast between −50 and −300 HU. Bowel lumen contents lower than −300 HU and higher than 100 HU again resulted in excessive under-estimation of bowel wall thickness, while bowel lumen contents between 0 and 100 HU resulted in excessive over-estimation of bowel wall thickness, the latter because the bowel wall CT attenuation was too close to that of the bowel lumen CT attenuation. For both experiments, the derangements in bowel wall thickness measurements were exacerbated with larger CT field of view, which resulted in larger voxel sizes and hence more volume averaging.

FIG. 17. DECT scans of patients with HSHBM of different concentrations and true density, shown at typical abdominal window/level setting of 400/40 HU. Top row: DECT scan obtained after oral administration of 350TA HSHBM 15% w/w shows CT attenuation of −200 HU in the stomach lumen (S) on the 120 kVp-like image (top left). The stomach wall is well depicted on the 120 kVp-like image since the stomach lumen contrast material is just below the HU of typical abdominal window/level required to be shown as pure black. Unfortunately, on the corresponding iodine map (top right), an unacceptable false iodine concentration of 2.1 mg iodine/mL, depicted as non-black gray signal (yellow arrow). is seen and may be mistaken for true iodine signal or may mask true iodine signal of adjacent iodine-contrast-enhanced structures on that image reconstruction. Bottom row: DECT scan obtained after oral administration of 270TA HSHBM 9% w/w shows CT attenuation of −180 HU in the stomach lumen (S) on the 120 kVp-like image (bottom left). Intravenous iodine contrast was also given for this scan. The stomach wall is again well depicted since the stomach lumen contrast material is just below the HU of typical abdominal window/level required to be shown as pure black. In addition, the corresponding iodine map (bottom right) shows no visible iodine signal in the stomach lumen (yellow arrow) and quantitative measurement shows less than 1 mg iodine/mL in the stomach lumen, indicating no substantial iodine signal artifact. The enhancement of the bowel wall with bright iodine contrast agent is beautifully shown. The informed formulation of oral contrast material with tailored HSHBM true density of appropriate concentration allows for appropriate DECT iodine map evaluation of the bowel wall.

FIG. 18. DECT scans of phantom with different hollow borosilicate microsphere suspensions, with 120 kVp-like reconstruction image (left image) and iodine map (right image), obtained on a General Electric 750 HD DECT scanner. A CT phantom was constructed with seven empty cylinders surrounded by lard, the latter to simulate human fat. Within the central cylinder are a vial of water (W) (center upper left) and one of iodine (I) (center lower right) that contains 2 mg I/mL in aqueous solution. The outer cylinders are filled with, starting from the top right and going clockwise, 270TA HSHBM 9% w/w (top right), 270TA HSHBM 7.5%, 270TA HSHBM 3%, 45P RHBM 30% w/w, 45P RHBM 20% w/w, and 350TA 15% w/w (top left). 270TA refers to a test article high silicon hollow borosilicate microparticle with true density 0.27 g/cm³, 45P refers to a regular hollow borosilicate microparticle with true density 0.45 g/cm³. 350TA refers to a test article high silicon hollow borosilicate microparticle with true density 0.35 g/cm³. The RHBM and HSHBM suspensions all appear dark on the 120 kVp-like CT image, with HU values of −178, −143, −68, −209, −158, and −213 HU, respectively, and the water is −9 HU and the 2 mg I/mL is 38 HU, as expected. Note that the 270TA HSHBM 9 and 7.5% w/w suspensions are darker than fat, and the 270TA HSHBM 3% w/w suspension are brighter than fat, which measures −95 HU, and can therefore be distinguished from fat on the 120 kVp image. On the iodine image reconstruction, the water measures −0.4 mg I/mL and the iodine measures 1.7 mg I/mL, as expected. The 270TA HSHBM suspensions each measure <0.7 mg I/mL, which is below the 0.8 mg I/mL threshold for confirmation of presence of iodine and would not be mistaken for iodine signal nor would it interfere with the detection of iodine of adjacent structures. The 270TA HSHBM measure 0.66, 0.45, and 0.15 mgI/mL for the 9, 7.5, and 3% w/w suspensions, respectively. However, the 45P RHBM 20 and 30% w/w suspensions show very bright signal even higher than that of the actual iodine solution vial, and show measured iodine concentrations of 3.1 and 3.9 mg I/mL, respectively. The 350TA HSHBM 15% w/w also shows artifactually high iodine concentration of 1.2 mg I/mL, which can be mistaken for actual iodine content or obscure the presence of iodine in adjacent structures.

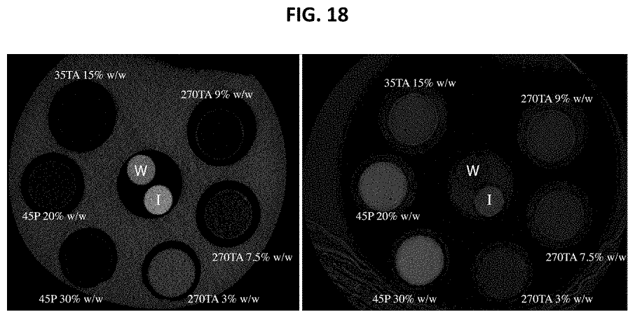

### II. DEFINITIONS

Unless defined otherwise, all technical and scientific terms used herein generally have the same meaning as commonly understood by one of ordinary skill in the art to which this invention belongs. Generally, the nomenclature used herein and the laboratory procedures in organic chemistry, pharmaceutically acceptable formulation, and medical imaging are those well-known and commonly employed in the art.

The articles “a” and “an” are used herein to refer to one or to more than one (i.e. to at least one) of the grammatical object of the article. By way of example, “an element” means one element or more than one element.

A “disease” is a state of health of an animal wherein the animal cannot maintain homeostasis, and wherein if the disease is not ameliorated then the animal's health continues to deteriorate.

“Contemporaneous” administration refers to use of a contrast agent in conjunction with a medical imaging procedure performed on a subject. As understood by one of skill in the art, contemporaneous administration of the contrast agent to the subject includes administration during or prior to the performance of the medical imaging procedure such that the contrast agent is visible in the medical image of the subject.

The term “half-life” or “t ½”, as used herein in the context of administering an enteric contrast medium of the invention to a patient, is defined as the time required for an effective enteric concentration of a drug in a patient to be reduced by one half. There may be more than one half-life associated with the contrast medium depending on multiple clearance mechanisms, redistribution, and other mechanisms well known in the art. For a hollow particle contrast material where the effectiveness of the contrast material depends on the integrity of the hollow void, the effective concentration is directly related to the concentration of the hollow void volume of the particle in the aqueous formulation in vivo. Further explanation of “half-life” is found in Pharmaceutical Biotechnology (1997, D F A Crommelin and R D Sindelar, eds., Harwood Publishers, Amsterdam, pp 101-120).

“Enteric contrast medium formulation” as herein used means, unless otherwise stated, a pharmaceutically acceptable liquid or paste formulation for administration to a subject, which comprises at least one enteric contrast medium, and with or without at least one pharmaceutically acceptable excipient suspending the medium, and which is prepared by dissolving, emulsifying, or suspending an enteric contrast medium as herein described, e.g. in the form of a powder, emulsion or mash, in a pharmaceutically acceptable vehicle, before use for administration to the subject. Preferably the suspending medium is water.

The term “hollow borosilicate microparticle,” abbreviated “HBM”, is used herein to describe a particle composed of borosilicate with outer diameter <500 microns and an internal void that may contain gas or partial vacuum. The term “regular HBM,” abbreviated “RHBM” is used herein to refer to a subset of HBM where the shell material is composed of about 60 to 85% SiO₂ and which has >2% oxides of atoms with atomic number greater than 10 (e.g., sodium oxide or aluminum oxide, etc). The term “high silicon HBM,” abbreviated “HSHBM” is used herein to refer to HBM where the shell material is composed of more than about 92% silicon dioxide and less than about 2% oxides of atoms with atomic number greater than 10.

The term “microsphere”, as used herein, refers to a subset of microparticles where the outer shape is spherical. The term “microparticle”, as used herein, includes microspheres and other particles with diameter in the range of from about 1 to about 800 microns.

The term “residence time”, as used herein in the context of administering an enteric contrast medium to a patient, is defined as the average time that the enteric contrast medium stays in the body of the patient after dosing.

The term “CT” refers to computed tomography imaging of any sort, including low dose, dual energy, multi-energy, and photon counting CT.

As used herein, “pharmaceutically acceptable carrier” includes any material, which when combined with the microspheres (particles) is compatible with the microspheres and tolerated by a subject to whom a pharmaceutical formulation incorporating the microspheres and the carrier is administered. Examples include, but are not limited to, any of the standard medical carriers such as a phosphate buffered saline solution, water, emulsions such as oil/water emulsion, and various types of wetting agents. Other carriers may also include sterile solutions. Typically, such carriers contain excipients such as starch, milk, sugar, sorbitol, methylcellulose, certain types of clay, gelatin, stearic acid or salts thereof, magnesium or calcium stearate, talc, vegetable fats or oils, gums, glycols, or other known excipients. Such carriers may also include flavor, texture, and color additives or other ingredients. Compositions comprising such carriers are formulated by well-known conventional methods.

As used herein, “administering” means oral administration, topical contact, intrarectal, intravenous, intraperitoneal, intralesional, intranasal or subcutaneous administration, intrathecal administration, or instillation into a surgically created pouch or surgically placed catheter or device, or the implantation of a slow-release device e.g., a mini-osmotic pump, to the subject.

The term “enteric contrast medium” as used herein is understood to mean a dry or unsuspended component or mixture of components comprising at least one X-ray absorbing substance and optionally at least one pharmaceutically acceptable excipient, which may itself include other components, e.g., taste-masking agents, antioxidants, wetting agents, flow or anti-caking agents, emulsifying agents, etc. The “dry suspension mixture” may subsequently be dissolved or suspended in a suspending medium to form the enteric contrast medium formulation of the invention. Terms such as “suspending medium” and “pharmaceutically acceptable excipient”, as used herein, refers to the medium in which the component(s) of the enteric contrast medium are emulsified or suspended.

The terms “coating” and “coated” as herein used are understood to include coatings which are biocompatible within an environment having an acidic, or a neutral, or a basic pH value.

The term “dark” as herein used to describe contrast material refers to having CT number less than about −20 HU.

The terms “particle”, “particles” and “microparticle(s) as used herein refers to free flowing substances of any shape which are larger than about 1 nm, such as crystals, beads (smooth, round or spherical particles), pellets, spheres, and granules. A particle may be a hollow bubble or contain multiple internal cavities. Exemplary specific sizes for the particles include from about 1 nm to about 500 microns, e.g., 1 micron to about 100 microns encompassing each single diameter value and each diameter range within the larger range across all endpoints; in various embodiments, the particles are larger than about 5 microns. Further useful particle sizes include, for example, from about 5 microns to about 100 microns, e.g., from about 20 microns to about 70 microns. A particle may contain gas or partial vacuum. A particle may be solid.

The term “suspending agent” as used herein refers to any convenient agent known in the art to be of use in forming and/or maintaining a suspension of a solid in a liquid (e.g., aqueous or oil). Exemplary suspending agents are selected from xanthan gum, gellan gum, guar gum, hydroxypropylmethyl cellulose, hydroxypropyl cellulose, polyvinyl pyrrolidone, alginates, and sodium carboxylmethylcellulose with xantham gum being preferred. Suspending agents may be employed in any useful amount. Exemplary useful amounts are within the range from about 0 to about 20% by weight of the powder formulation, and from about 0 to about 10% by weight of the oral suspension. Exemplary suspending agents are incorporated into the formulation in an amount of from about 0.1% to about 20%, e.g., from about 0.5% to about 15%, e.g., from about 1% to about 10%, e.g., from about 3% to about 8%.

“Stable” in the context of the invention refers to suspensions that do not significantly separate into their components as different phases or layers between manufacture of the suspension and the time of medical image acquisition after its administration to a subject in an imaging study or from the time of suspension of the agent in the pharmaceutically acceptable carrier and the time of medical image acquisition after its administration to a subject in an imaging study. By way of non-limiting illustration, imaging occurs after a period of from about 1 minute to about 180 minutes after ingestion of the contrast agent for imaging of the esophagus, stomach, or small bowel, and from at least about 1 hour to about 2 days after ingestion of the contrast agent for imaging of the colon, during which time the suspensions of the invention do not significantly separate into their components as different layers.

“True Density”, as this term is used herein, refers to the mass of the material per volume that it occupies, excluding surrounding gas that is in free communication with the atmosphere, such as may be measured using a gas pycnometer. “Mean true density”, as this term is used herein, refers to the mass of a given sample of material per the volume that it occupies, excluding surrounding gas and gas between particles of the material that is in free communication with the atmosphere. Mean true density may be measured using a gas pycnometer.

The term “hollow” as used herein refers to gas or vacuum that is confined and highly restricted from communication with the external environment such that a minimal amount of the gas or vacuum is released from the confined space, and a minimal amount of fluid enters the confined space, during the expected residence time of biological use. Any gas within the hollow borosilicate microparticle may be at a lower, same, or higher pressure than the surrounding atmosphere or suspending liquid vehicle.

The term “dark contrast” as used herein refers to material producing lower CT number signal than water (CT number <−20 HU).

“An unpleasant taste” as used herein means that a majority of human patients judge said enteric contrast medium comprised as having an unpleasant taste when ingested.

### III. EXEMPLARY EMBODIMENTS

### A. Compositions

In various embodiments, the present invention provides enteric or non-vascular contrast agents that produce dark CT numbers lower than about −20 HU at CT imaging. In various embodiments, the present invention provides contrast agents containing hollow borosilicate microparticles with overall CT numbers of the formulation from about −20 to about −70 HU which is below the CT number of water and higher than the CT number of fat. In various embodiments, the present invention provides contrast agents containing hollow borosilicate microparticles with overall CT numbers of the formulation from about −160 to about −300 HU which is below the range of CT numbers that would be displayed as black on standard CT images viewed with standard soft tissue window and level viewing settings (window and level of 400 and 40), yet not so negative in CT number as to cause excessive loss of visibility of the bowel wall. Exemplary materials include hollow borosilicate microparticles with shell material containing greater than about 90% SiO₂ and < about 10% oxides of other non-silicon atoms with z>10.

In various embodiments, the shell of the particles of the contrast media of the invention is a formed from predominantly SiO₂. In various embodiments, the shell of the particles of the contrast media contains over about 90% SiO₂. In various embodiments, the shell of the particles of the contrast media contains over about 90% SiO₂ and less than about 5% B₂O₃ and less than about 4% oxides of atoms with atomic number greater than 10.

In various embodiments, the true density of the particles is greater than about 0.05 g/cm³. In various embodiments, the true density of the particles of the contrast medium of the invention is at least about 0.1, at least about 0.2, or at least about 0.25 g/cm³. In various embodiments, the true density of the particles is less than 0.5 g/cm³, less than 0.4 g/cm³, or less than 0.35 g/cm³

In various embodiments, the interior space of the particle is at least partially gas filled as discussed herein. When the interior of the particle is at least partially filled with a gas other than air, the gas is preferably not a hydrocarbon, fluorocarbon, sulfur compound or a hydrofluorocarbon. In various embodiments, the gas is an elemental gas. In various embodiments, the gas contains carbon dioxide, oxygen, nitrogen, air or a combination thereof.

Exemplary particles of the invention are low in true density yet maintain substantial isostatic crush strength and do not break with medical ultrasound imaging forces so that the hollow void is not readily destroyed by physiologic forces within the imaged organism. Exemplary particles of the invention show no more than 5% loss of the hollow volume when subjected to isostatic pressures of 500 psi. Exemplary particles of the invention do not show more than about 2% loss of the hollow volume when subjected to ultrasound imaging and pulses at medical imaging for about 15 minutes, including pulses used to burst conventional ultrasound bubble contrast materials.

Exemplary contrast media of the invention decrease the CT number of the lumen of the gastrointestinal tract or other body cavity to below that of pure black on soft tissue window/level viewing settings. Exemplary contrast media of the invention decrease the CT number of the lumen of the gastrointestinal tract or other body cavity to between that of water and fat at CT imaging.

The contrast agents of our invention can provide improved CT imaging applications with one or more of the following benefits:
1) bowel lumen or non-vascular structures containing the contrast material of the invention can be more easily differentiated from soft tissue than if filled by currently available contrast material.
2) bowel or non-vascular structures can be filled by contrast materials of the invention and be distinguished at CT imaging from vascular structures or soft tissue enhanced by intravenous positive CT contrast agents.
3) enteric or nonvascular structures can be opacified with contrast of the invention for CT imaging without interfering with the assessment of intravascular positive contrast material related mural enhancement of those structures (bowel wall, bladder wall, other walls, including associated disease such as inflammation or neoplasms) based on CT signal at single energy spectrum CT or by relative low to high energy X-ray attenuation ratio at dual energy or spectral CT.

In various embodiments, the invention provides enteric contrast agents hollow borosilicate microparticles. In various embodiments, the contrast agent can be selected to provide a CT number between −20 and −70 HU. In various embodiments, the contrast agent can be selected to provide a CT number between −160 and −300 HU. In various embodiments, the contrast agent formulation includes hollow borosilicate microparticles in an aqueous media.

In various embodiments, the shell material of the hollow borosilicate microparticles includes from about 0.3 to about 8%, e.g., from about 0.5 to about 7%, from about 1% to about 6%, e.g., from about 2% to about 4% boron trioxide

In an exemplary embodiment, the hollow lumen contents of the particle is carbon dioxide, or is largely oxygen, nitrogen and carbon dioxide. In various embodiments, the contents of the hollow particle does not contain sulfur, or is essentially devoid of sulfur.

In an exemplary embodiment, the hollow borosilicate microparticle has a mean true density of from about 0.1 to about 0.4 g/cm³. In an exemplary embodiment, the hollow borosilicate microparticle has a mean true density of from about 0.2 to about 0.35 g/cm³.

One or two or more hollow borosilicate microparticle types may be used together.

Any useful suspending agent or combination of suspending agents can be utilized in the formulations of the invention. In various embodiments, the suspending agent is thixotropic and forms a gel-like medium at rest but a liquid with agitation.

In an exemplary embodiment, the enteric contrast medium is formulated into a pharmaceutically acceptable carrier in which the HBM is suspended.

In an exemplary embodiment, the hollow borosilicate microparticle is coated to provide useful properties for the contrast material, such as improved suspension in media, increased true density, or alter the CT number or 80:140 kVp CT number ratio, or alter the apparent iodine concentration at CT or DECT or multienergy CT or photon counting CT imaging.

In an exemplary embodiment, the coating comprises an organic molecule with a molecular weight of less than about 3 kd, less than about 2 kd or less than about 1.5 kd. In an exemplary embodiment, the coating comprises an organic molecule with a molecular weight of less than about 3 kd, less than about 2 kd or less than about 1.5 kd, which is a member selected from an organic acid (or alcohol, amine) and its derivatives or analogs, an oligosaccharide and a combination thereof.

In an exemplary embodiment, the coating is a protein, e.g., albumin.

In various embodiments, the particles of the invention are coated with a biocompatible coating. Appropriate coatings are known in the art and it is within the abilities of one of skill in the art to select an appropriate coating for a particular formulation and/or application. (See, for example, Yeh B M, Fu Y, Desai T, WO 2014145509 A1).

The suspended phase of formulations of the invention can include particles of any useful size and size range. Exemplary specific sizes for the particles include from about 1 nm to about 500 microns, e.g., 1 micron to about 100 microns encompassing each single diameter value and each diameter range within the larger range across all endpoints; in various embodiments, the particles are larger than about 5 microns. Further useful particle sizes include, for example, from about 5 microns to about 100 microns, e.g., from about 20 microns to about 70 microns.

The formulations of the invention can include a single enteric contrast medium or two or more enteric contrast media. The media can be present in similar or different concentrations according to any useful measure of concentration. An exemplary embodiment includes different concentrations of one or more particles or soluble agents such that each contributes substantially to the x-ray attenuation, relative to that of water, in the overall contrast formulation. Thus, in various embodiments, from about 1% (w/w, expressed as a weight percent, e.g., about 1 gram of contrast agent particle contained in about 100 grams of total contrast formulation) to about 10% (w/w) of the weight of said formulation is said particles. In an exemplary embodiment, the formulation includes about 3% (w/w) to about 9% (w/w) of the particles. In an exemplary embodiment, the formulation includes about 1% to about 3% (w/w) of the particles.

In an exemplary embodiment, the invention provides a formulation comprising at least about 1%, e.g., at least about 2% but not more than about 10% of said hollow borosilicate particle.

The formulations of the invention include a population of hollow borosilicate microparticle of the invention suspended in a pharmaceutically acceptable vehicle. The vehicle includes any other useful component. For example, in some embodiments, the vehicle comprises an aqueous medium, and it further comprises an additive to impart a second property to the formulation, for example, retard dehydration of said formulation in the bowel, provide flavoring, stabilize the suspension, enhance flowability of the suspension, thicken the suspension, provide pH buffering and a combination thereof.

Within the scope of the invention are formulations designed for single dosage administration. These unit dosage formats contain a sufficient amount of the formulation of the invention to provide detectable contrast in a subject to whom they are administered. In an exemplary embodiment, the unit dosage formulation includes a container holding sufficient enteric contrast medium to enhance, in a diagnostically meaningful manner, a diagnostic image of a subject to whom the unit dosage has been administered. The container can be a vial, an infusion bag, bottle, sachet, or any other appropriate vessel. The enteric contrast medium may be in the form of a preformulated liquid, a concentrate, or powder. In an exemplary embodiment, the subject weighs about 70 kg. In an exemplary embodiment the image is measured through the abdomen of the subject, the pelvis of the subject, or a combination thereof.

In various embodiments, the unit dosage formulation includes from about 800 to about 1500 mL of the contrast agent per adult human dose, which may be divided into smaller containers such as from about 300 to about 600 mL in size. In an exemplary embodiment, the enteric contrast medium formulation is a unit dosage formulation of from about 50 to about 100 mL. In an exemplary embodiment, the enteric contrast medium formulation is a unit dosage formulation of from about 100 mL to about 800 mL.

Any of the formulations described herein can be formulated and utilized for administration through any of a variety of routes. Exemplary routes of administration include oral, rectal, intravaginal, intravascular, intrathecal, intravesicular, and the like.

Low concentrations of HBM contrast materials have not been described for use with CT imaging as a contrast material. In an exemplary embodiment, the HBM in the formulation are of low concentration, e.g., about 0.5% (w/w) to about 10% (w/w), e.g., from about 1% to about 4%, e.g., from about 1.5% to about 3% of the formulation.

In various embodiments, the enteric contrast medium of the invention and preferably its formulation exhibits chemical stability across a wide pH range (e.g., from about 1.5 to about 10). The stomach exposes enteric contents to low pH of 1.5 and bile and small bowel may expose enteric contents to high pH of up to 10. Physicochemical stability is a critical component of safety and helps minimize the risk of reactions or adverse events. Adverse reactions may occur if excessive dissolution or degradation of the materials were to occur in the gastrointestinal tract, or if the breakdown products are potentially toxic.

In various embodiments, the invention provides an enteric contrast medium and a formulation of a contrast medium with a t_{1/2} that is sufficiently long to allow the completion of an imaging experiment with the concentration of HBM remaining sufficiently high within the anatomy of interest. In various embodiments, the invention provides an enteric contrast medium and a formulation having an in vivo residence time that is sufficiently short to allow essentially all of the administered HBM to be eliminated from the body of the subject before being altered (metabolized, hydrolyzed, oxidized, etc.) by the subject's body.

In various embodiments, the small bowel enteric transit time of the formulation is less than 12 hours in normal subjects. In an exemplary embodiment, the formulation includes polyethylene glycol or sugar alcohols such as sorbitol, mannitol, and xylitol or both to accelerate enteric transit times.

In an exemplary embodiment, the invention provides an enteric contrast medium that dissolves slowly such that the majority of the administered HBM particles are eliminated via the gastrointestinal tract prior to being altered by the subject's body, and a dissolved or altered portion is excreted by the urinary tract.

The pharmaceutically acceptable formulation of the present invention may optionally include excipients and other ingredients such as one or more sweeteners, flavors and/or additional taste modifiers to mask a bitter or unpleasant taste, suspending agents, glidants, antioxidants, preservatives and other conventional excipients as needed.

The suspension of the invention may optionally include one or more antioxidants, if necessary, taste modifiers, sweeteners, glidants, suspending agents, and preservatives.

As will be appreciated, the above optional ingredients may be added to the powder formulation of the invention, or to the oral suspension of the invention.

Antioxidants suitable for use herein include any convenient agents known in the art for this purpose such as sodium metabisulfite, sodium bisulfite, cysteine hydrochloride, citric acid, succinic acid, ascorbic acid, sodium ascorbate, fumaric acid, tartaric acid, maleic acid, malic acid, EDTA with sodium metabisulfite or sodium bisulfite being preferred.

Antioxidants may be employed in an amount which will protect the formulation from oxidation as will be apparent to one skilled in the art.

Sweeteners for use in the formulations of the invention may be any convenient agents known in the art for this purpose and may be selected from any compatible sweetener groups such as natural sweeteners like sucrose, fructose, dextrose, xylitol, sorbitol, or manitol, as well as artificial sweeteners such as aspartame, acesulfame K and sucralose. Sucralose and sorbitol are preferred sweeteners.

Flavors and flavor modifiers or taste modifiers can also be used to further improve the taste and can be any convenient agents known in the art for this purpose and include, but are not limited to, orange flavor, vanilla flavor, toffee flavor, licorice flavor, orange vanilla flavor, creme de mint, cherry flavor, cherry vanilla flavor, berry mix flavor, passion fruit flavor, pear flavor, strawberry flavor, mandarin orange flavor, bubble gum flavor, tropical punch flavor, juicy compound for grape, grape flavor, artificial grape flavor, grape bubble gum flavor, tutti-frutti-flavor, citrus flavor, lemon flavor, chocolate flavor, coffee flavor, matcha flavor, and combinations thereof.

Suspending agents can be any convenient agents known in the art for this purpose and can be selected from xanthan gum, gellan gum, guar gum, hydroxypropylmethyl cellulose, hydroxypropyl cellulose, polyvinyl pyrrolidone, alginates, sodium carboxylmethylcellulose and combinations thereof, with xanthan gum being preferred in some embodiments.

Preservatives can be any convenient agents known in the art for this purpose and can be selected from the group consisting of any compound compatible with drug actives such as methylparaben and propylparaben, benzoic acid, sodium benzoate, potassium sorbate, and combinations thereof, with methylparaben being preferred in some embodiments.

The invention also provides kits for use in a clinical and/or research setting. An exemplary kit includes: (a) a first vial containing the enteric contrast medium of the invention; (b) a second vial containing a suspension agent; and (c) directions for using and/or formulating the enteric contrast medium as a suspension. In various embodiments, the kit further comprises another vial containing a second contrast medium; and directions for administering and/or formulating the first and second enteric contrast medium in a clinical or research setting.

### B. Methods

The invention also provides methods of utilizing the formulations of the invention to acquire and enhance clinically meaningful CT images from a subject to whom the formulation of the invention is administered. The method includes, administering to the subject a diagnostically effective amount of said enteric contrast medium formulation of the invention; and acquiring the CT images of the subject.

The invention also provides methods of utilizing the formulations of the invention concurrent with additional CT contrast agents such as iodinated agents which may be injected or ingested to acquire and enhance clinically meaningful CT images from a subject to whom the formulation of the invention is administered. The method includes administering to the subject a diagnostically effective amount of the enteric contrast medium formulation of the invention then injecting another CT contrast agent then acquiring the CT images of the subject. CT images may be acquired on conventional CT scanners or with dual energy CT, multi-energy CT, or photon counting CT scanners.

In an exemplary embodiment, the invention provides a contrast enhanced CT image of a subject through a region of the subject in which the enteric contrast medium of the invention is distributed. The said contrast enhanced CT image of the invention may be a conventional single energy spectrum CT image, or may be dual energy, multienergy, or photon counting CT images with or without associated CT image reformations that exploit the dual energy, multienergy, or photon counting CT technology. In an exemplary embodiment, the CT image of the invention provides a iodine image or iodine map through a region of the subject in which the enteric contrast medium of the invention is distributed concurrent with iodinated contrast material.

The image of the invention, and those acquired by the method of the invention utilize a contrast medium of the invention. The image is taken through any section of the body of the subject. In an exemplary method, the image is through the abdomen and/or pelvis of the subject.

The following Examples are offered to illustrate exemplary embodiments of the invention and do not define or limit its scope.

### EXAMPLES

### Example 1

Hollow borosilicate glass microparticle “Test Article” (TA) with shell material composed of 95% SiO₂, 2% B₂O₃ and less than 3% oxides with atomic number larger than 10 (such as sodium, aluminum, magnesium, and calcium oxides) were formed with true gravity of 0.35 g/cm³ as determined by helium gas pycnometry. The formation of the hollow borosilicate microparticles did not involve sulfur. This Test Article was named 350TA. The shell composition was confirmed by X-ray fluorescence. The 350TA was then suspended in an aqueous solution as 30%, 20%, 15%, 10%, 5%, and 3% w/w suspensions of Test Article, with the water solution containing 0.2 to 0.4% w/w xanthan gum and 3% w/w sorbitol.

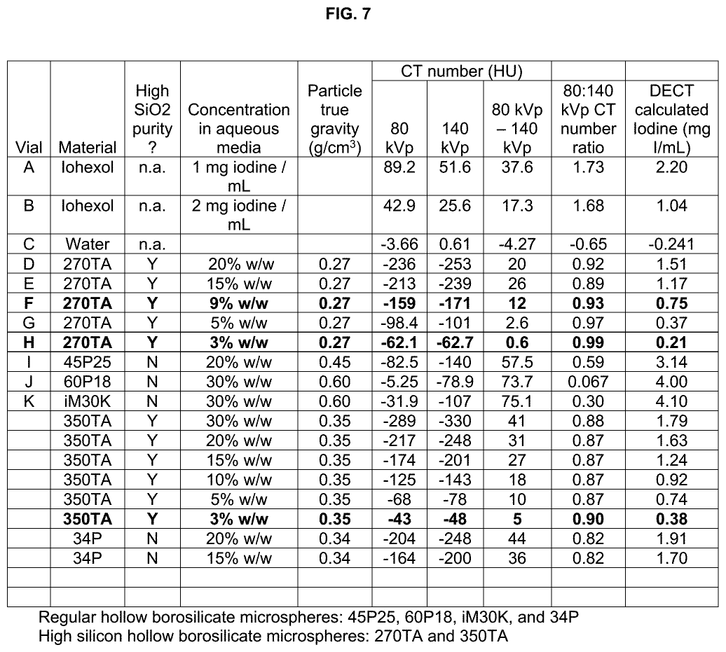

The four 350TA suspensions were scanned in vitro on a dual energy CT scanner which showed the results shown on FIG. 7. Of these 350TA suspensions, the 3% and 10% w/w 350TA formulation provided CT number range (−43 to −48 HU and −125 to −143 HU, respectively) sufficient to distinguish from both water/soft tissue (−20 to 50 HU) and fat (−70 to −120 HU) at conventional CT while also providing iodine map calculated iodine concentration (0.38 and 0.92 mg I/mL, respectively) less than that of detectable iodine (1 mgI/mL). The other 350TA formulations either showed a calculated iodine map iodine concentration greater than 1 mg I/mL (the 15%, 20%, and 30% w/w 350TA formulations) or showed CT numbers that may overlap that of normal fat (5% w/w 350TA formulation). Note that the accuracy of iodine map calculated iodine concentrations in a small phantom/in vitro experiment shows much less noise than would be expected in vivo due to larger patient size and visceral movement. More noise and lower detection limit of iodine concentration is expected in vivo

The formulations were re-tested with additional excipients including up to 4% flavoring and 2% sucralose and preservatives with similar CT results.

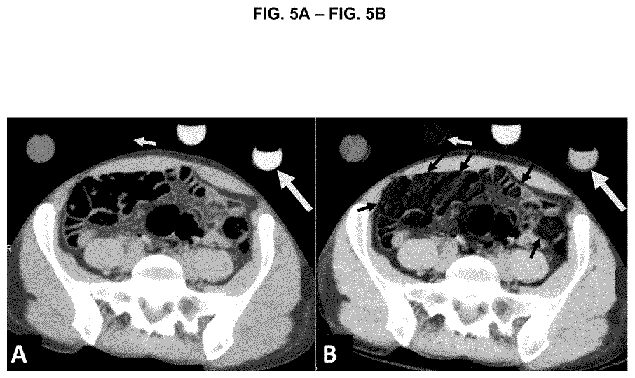

The 15% w/w 350TA suspension with 4% flavoring and 2% sucralose was given by the oral route to healthy volunteers. Prior to and after consumption of the 350TA suspension, the volunteers were scanned on a DECT scanner. The volumes of the 350TA suspension ranged from 400 to 2000 mL. The bowel was found to be marked by the 350TA suspension with CT number average of −170 HU, which allowed ready delineation from bodily fat in the vast majority of cases. However, DECT iodine map reconstructions showed undesired low level calculated iodine concentrations similar to or greater than background soft tissues such as muscle (FIG. 5). No uptake of silicon was seen in the blood and no pattern of increased urine silicon above background levels was seen of the volunteers at 1 hour, 4 hours, and 1 day after consumption of the 270TA formulation. No serious adverse events were noted.

### Example 2

Hollow borosilicate microparticle “Test Article” (TA) with true gravity of 0.27 g/cm³ and shell material composed of 95% SiO₂, 2% B₂O₃ and less than 2% oxides with atomic number larger than 10 (e.g. sodium, aluminum, magnesium, calcium oxide). The formation of the hollow borosilicate microparticles did not involve sulfur. The true gravity was confirmed by helium gas pycnometry. This Test Article was named 270TA. The shell composition was confirmed by X-ray fluorescence. The 270TA was then suspended in an aqueous solution as 20%, 15%, 9%, 5%, and 3% w/w suspensions of Test Article, with the water solution containing 0.2 to 0.5% w/w xanthan gum and 3% w/w sorbitol.

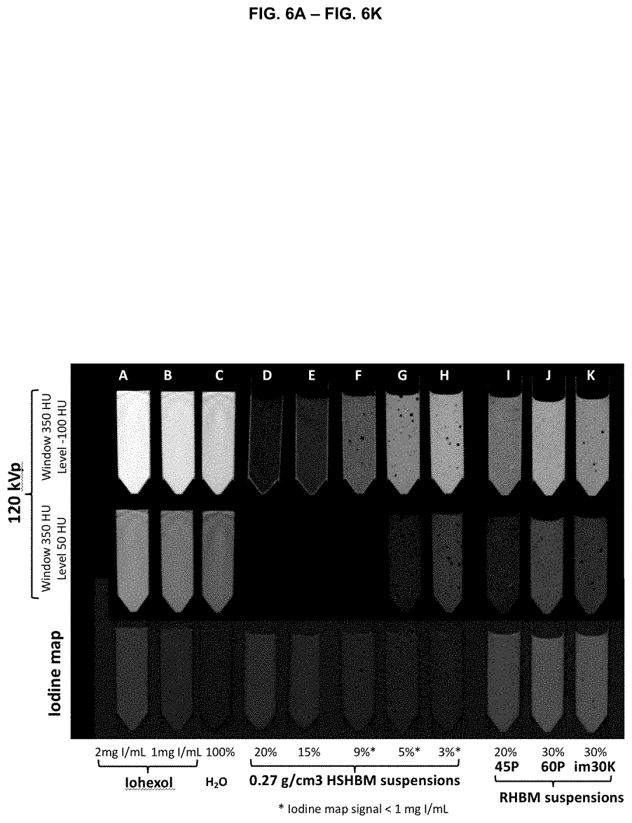

The four 270TA suspensions were scanned in vitro on a dual energy CT scanner which showed the results shown on FIGS. 6 and 7. Of these 270TA suspensions, the 3% and 9% w/w 270TA formulation provided CT number range (−62.1 to −62.7 HU and −159 to −171 HU, respectively) sufficient to distinguish from both water/soft tissue (−20 to 50 HU) and fat (−70 to −120 HU) at conventional CT, and also provided iodine map calculated iodine concentrations (0.21 and 0.75 mg I/mL, respectively) less than that of detectable iodine (1 mgI/mL). The other 270TA formulations either showed a calculated iodine map iodine concentration greater than 1 mg I/mL (the 10%, 15%, and 20% w/w 270TA formulations) or showed CT numbers that may overlap that of normal fat (5% w/w 270TA formulation). Note that the accuracy of iodine map calculated iodine concentrations in a small phantom/in vitro experiment shows much less noise than would be expected in vivo due to larger patient size and visceral movement.

The 270TA formulations were re-tested with additional excipients including up to 4% flavoring and 2% sucralose and preservatives with similar CT results.

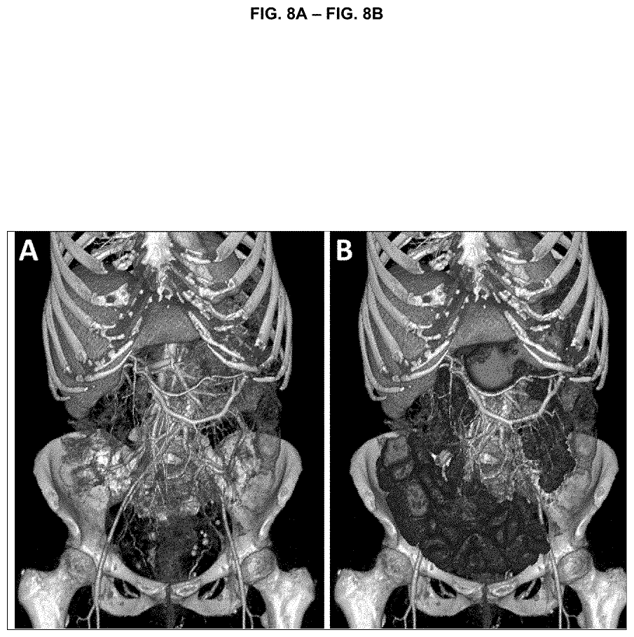

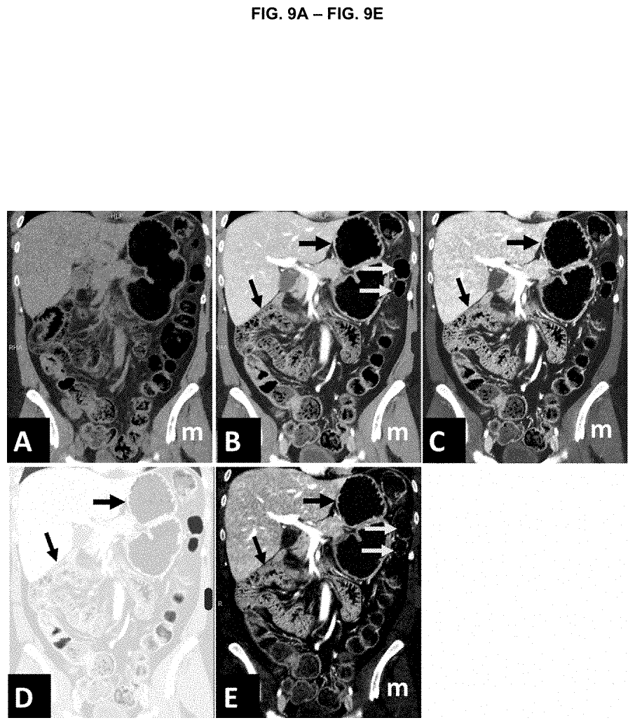

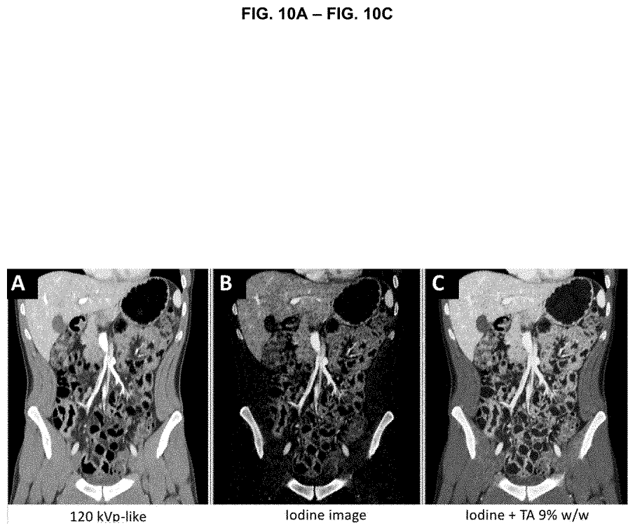

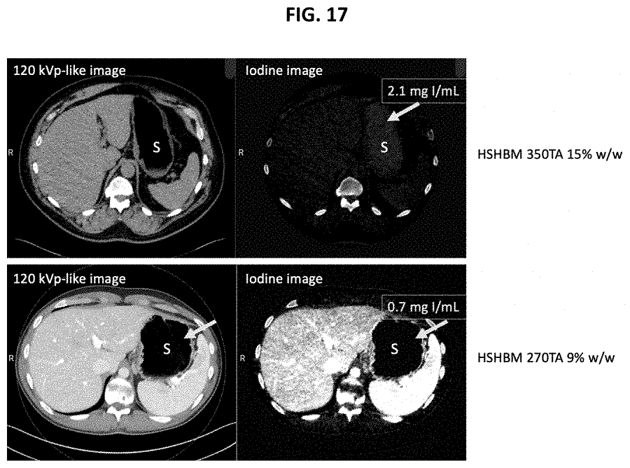

Formulations of 9% w/w 270TA with 0.3% xanthan gum, 3% sorbitol, 4% flavoring and 2% sucralose were administered orally as 1200 mL doses to 32 patient volunteers. The volunteers were imaged at CT prior to and after consumption of the 270TA formulation. The CT scan after consumption of the 270TA formulation utilized dual energy CT and injected intravenous contrast material. Example images are shown in FIGS. 7, 9 and 10. The bowel lumen was marked and distended by the 270TA formulation with average CT number of −180 HU in the stomach and −220 HU in the distal ileum and cecum, which allowed the 270TA contrast material formulation to be readily delineated from iodinated contrast material, soft tissues, and fat at conventional CT images (FIGS. 8 and 9). At DECT iodine map image reconstructions, no visible calculated unwanted false iodine signal above 1 mg I/mL was seen in the bowel lumen (FIGS. 9, 10, and 17). The DECT images are able to clearly delineate true iodine signal from that of the 270TA signal and can delineate the 270TA signal also from biological fluid such as that of the gallbladder and bladder, and from fat and muscle (FIGS. 9 and 10)

No uptake of silicon was seen in the blood and no pattern of increased urine silicon above background levels was seen of the volunteers who consumed the 270TA formulation at 1 hour, 4 hours, and 1 day after consumption of the 270TA formulation. No serious adverse events were noted.

### Example 3

Exposure of patients to sulfur may result in unwanted reactions, and so the amount of sulfur in a drug or medical device should be minimized. The total sulfur content between RHBM (regular hollow borosilicate microparticle, including iM30K, 45P25, and 60P18) and HSHBM (high-silicon hollow borosilicate glass microparticles, including with true density of 0.27 and 0.35) as measured via a Leco sulfur analyzer. Method involves heating HBM sample to 1350° C. in an induction furnace while passing a stream of oxygen through the sample. Sulfur dioxide released from sample is measured by IR detection, and total sulfur content is reported. The tested HSHBM each showed sulfur content below detectable (<0.01%) while the RHBMs iM30I, 45P25, and 60P18 showed sulfur contents of 0.08%, 0.15%, and 0.16%, respectively.

The present invention has been illustrated by reference to various exemplary embodiments and examples. As will be apparent to those of skill in the art other embodiments and variations of this invention may be devised by others skilled in the art without departing from the true spirit and scope of the invention. The appended claims are to be construed to include all such embodiments and equivalent variations.

The disclosures of each and every patent, patent application, and publication cited herein are hereby incorporated herein by reference in their entirety.

### Example 4

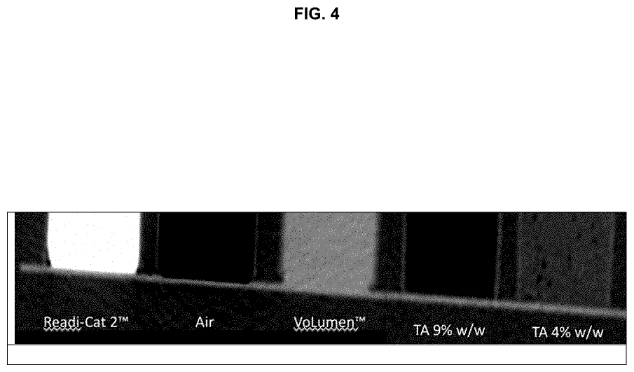

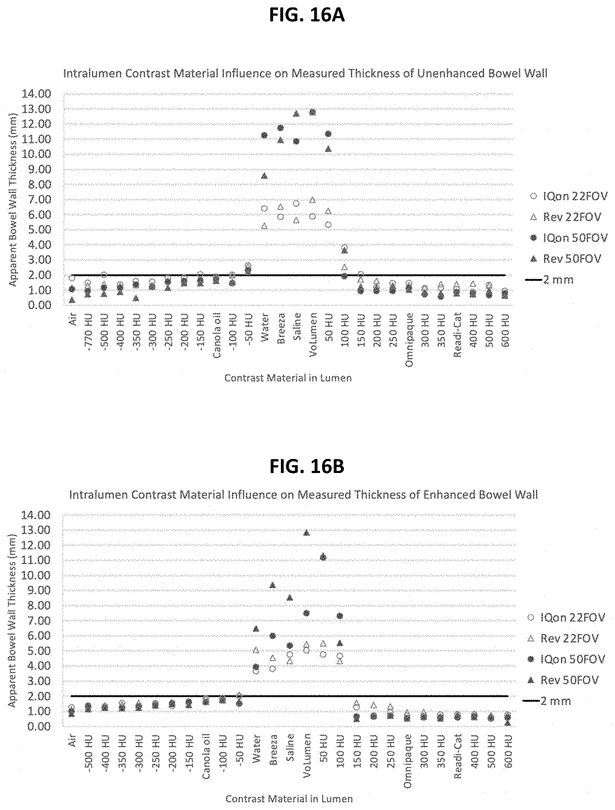

CT phantoms were constructed with open-ended plastic cylinders attached to 2.0 mm thick plastic sheets that were engineered to match the CT number of unenhanced bowel wall which is 40 HU (FIGS. 4 and 16A), or intravenous iodine contrast-enhanced bowel wall which was chosen to be about 165 HU (FIG. 16B). The phantoms was partially submerged in canola oil (FIG. 4) or lard (FIGS. 16A and 16B) to simulate human fat that typically surrounds bowel in the abdomen, then the cylinders were filled with a range of contrast media with a range of CT numbers. The measured thickness of the simulated bowel walls at CT most closely and consistently approximated the true 2.0 mm thickness for the contrast media with CT numbers between −50 HU to −300 HU, regardless of whether or not the engineered bowel wall phantom was unenhanced or enhanced with iodine intravenous contrast material. These superior-performing exemplary contrast media had HSHBGM concentrations of 2% to 25%.

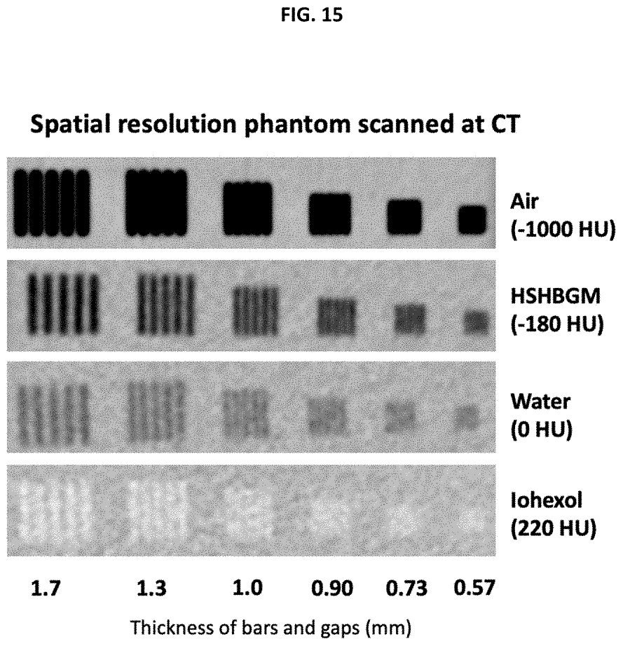

Spatial resolution phantoms constructed of plastic that simulated a range of thicknesses of bowel wall folds enhanced with iodine intravenous contrast material were filled with different commercial oral contrast media and exemplary HSHBGM contrast medium, the latter of which had CT number of −180 HU (FIG. 15). At CT imaging at 120 kVp using standard CT scan parameters, and displayed at typical abdominal window/level settings of 400/40 HU, the CT images obtained with the exemplary HSHBGM contrast medium showed better spatial resolution than that seen with the commercial CT oral contrast media.

### REFERENCES

1. Kreit E, Mäthger L M, Hanlon R T, et al. Biological versus electronic adaptive coloration: How can one inform the other? J R Soc Interface. 2013; 10(78). doi:10.1098/rsif.2012.0601
2. Spilde J M, Lee J, Chosy S G, Krupinski E A, Kuhlman J E, Yandow D R. Evaluation of an experimental low-attenuation gastrointestinal contrast agent for CT imaging of intestinal ischernia in an animal model. Acad Radiol. 1999; 6(2):94-101. doi:10.1016/s1076-6332(99)80488-7
3. Wei X, Zhu J, Gong H, Xu J, Xu Y. A novel foam fluid negative contrast medium for clear visualization of the colon wall in CT imaging. Contrast Media Mol Imaging. 2011; 6(6):465-473. doi:10.1002/cmmi.446
4. Leander P, Adnerhill I, Böök O, Casal-Dujat L, Stathis G, Fork T. A novel food-based foam as oral contrast agent with negative Hounsfield units for demarcation of small bowel loops on abdominal CT: tolerability and bowel distension in 25 volunteers. Acta radiol. 2020. doi:10.1177/0284185120973620
5. Ramsay D W, Markham D H, Morgan B, Rodgers P M, Liddicoat A J. The use of dilute calogen® as a fat density oral contrast medium in upper abdominal computed tomography, compared with the use of water and positive oral contrast media. Clin Radiol. 2001; 56(8):670-673. doi:10.1053/crad.2001.0772
6. Raptopoulos V, Davis M A, Davidoff A, et al. Fat-density oral contrast agent for abdominal CT. Radiology. 1987; 164(3):653-656. doi:10.1148/radiology.164.3.3615862
7. Raptopoulos V, Davidoff A, Karellas A, Davis M A, Coolbaugh B L, Smith E H. CT of the pancreas with a fat-density oral contrast regimen. Am J Roentgenol. 1988; 150(6):1303-1306. doi:10.2214/ajr.150.6.1303
8. Raptopoulos V, Davis M A, Smith E H. Imaging of the Bowel Wall Computed Tomography and Fat Density Oral-Contrast Agent in an Animal Model. Invest Radiol. 1986; 21(11):947-850.
9. Zwaan M, Gmelin E, Borgis K J, Rinast E. Non-absorbable fat-dense oral contrast agent for abdominal computed tomography. Eur J Radiol. 1992; 14(3):189-191. doi:10.1016/0720-048X(92)90084-M

1. An enteric contrast medium formulation which is formulated for oral delivery to a subject contemporaneous with a medical CT imaging procedure performed on the abdomen or pelvis of said subject, said formulation comprising:
an enteric contrast medium comprising a suspension of a hollow borosilicate microparticle with shell containing more than 90% SiO₂ and less than 8% non-silicon oxides of z>10 and an aqueous vehicle component, with said aqueous component which is a pharmaceutically acceptable aqueous vehicle,
where at CT imaging the contrast medium formulation gives CT number between −70 to −20 HU or between −160 and −300 HU when imaged at 120 kVp.

2. An enteric contrast medium formulation which is formulated for oral delivery to a subject contemporaneous with a CT imaging procedure performed on the abdomen or pelvis of said subject, said formulation comprising a hollow borosilicate microparticle with shell material and CT imaging characteristics described in claim 1.

3. The enteric contrast medium formulation of claim 1, wherein said formulation is a unit dosage formulation comprising a diagnostically effective amount of said enteric contrast medium.

4. The enteric contrast medium formulation of claim 1, wherein said formulation is a unit dosage formulation of from about 800 mL to about 2000 mL per adult human dose, which may be divided into smaller containers such as 400 mL to 600 mL in volume.

5. The enteric contrast medium formulation of claim 1 wherein said formulation is a unit dosage formulation of from about 50 to about 100 mL in volume.

6. The enteric contrast medium formulation of claim 1, wherein formulation is a unit dosage formulation of from about 100 mL to about 800 mL in volume.

7. The enteric contrast medium formulation of claim 1, wherein the apparent iodine concentration of the formulation at dual energy CT, multienergy CT, or photon counting CT is <1.0 mg iodine/mL at iodine image reconstructions.

8. The enteric contrast medium formulation of claim 1, wherein said hollow borosilicate microparticle does not contain sulfur.

9. The enteric contrast medium formulation of claim 1, wherein said formulation comprises at least about 0.5% weight/weight percentage of said hollow borosilicate microparticle (from about 1.0% to about 15% in terms of weight/weight percentage in aqueous formulation).

10. The enteric contrast medium formulation of claim 1, wherein said suspension agent is selected from xanthan gum, guar gum, gellan gum, hydroxypropylmethyl cellulose, hydroxypropyl cellulose, polyvinyl pyrrolidone, alginates, polyethylene glycol chains, and sodium carboxylmethylcellulose.

11. The enteric contrast medium formulation of claim 1, wherein said formulation is a unit dosage formulation and it contains more than about 3 g of said hollow borosilicate microparticle per 400 mL of aqueous suspension.

12. The enteric contrast medium formulation of claim 1, wherein said suspension agent comprises xanthan gum or gellan gum.

13. The enteric contrast medium formulation of claim 1, wherein said pharmaceutically acceptable vehicle further comprises an additive to retard dehydration of said formulation in the bowel, a flavoring agent, a thickening agent, a flow agent, a pH buffer, a laxative, an osmolality-adjusting agent, a preservative, and a combination thereof.

14. The enteric contrast medium formulation of claim 1, wherein the hollow borosilicate microparticle has a true gravity from about 0.15 to about 0.40.

15. The enteric contrast medium formulation of claim 1, wherein the hollow borosilicate microparticle has a mean diameter of from about 10 to about 60 micrometers.

16. The enteric contrast medium formulation of claim 1, wherein the hollow borosilicate microparticle has a mean diameter of from about 60 to about 200 micrometers.

17. The enteric contrast medium formulation of claim 1, wherein said enteric contrast medium is provided in powdered or other concentrated form to be mixed with water or other said acceptable medical aqueous vehicle near the time of administration for CT imaging, together with instructions for preparing an administrable enteric contrast medium and, optionally, one or more device for administering said administratable enteric contrast medium to a subject.

18. The enteric contrast medium formulation of claim 1, wherein the hollow borosilicate microparticle makes up 1.0% or more of the weight of the formulation.

19. A method of acquiring contrast enhanced CT imaging of a subject, said method comprising: administering to said subject a diagnostically effective amount of said enteric contrast medium formulation of claim 1.

20. The method according to claim 19, wherein said CT projection data are reconstructed into a CT image.

21. The method according to claim 19, wherein said CT images are used to distinguish said enteric contrast medium formulation from other materials or tissues in the abdomen or pelvis.

22. The method according to claim 19, wherein machine learning or artificial intelligence is used to segment the anatomy within said CT images.

23. The method according to claim 19 wherein computer aided diagnosis, machine learning, or artificial intelligence is used to identify CT findings within said CT images

24. The method according to claim 19 wherein computer aided diagnosis, machine learning, or artificial intelligence is used to identify diagnoses within said CT images

25. The method according to claim 19, wherein said image is an image of a region selected from the abdomen and pelvis of said subject.

26. The method of claim 19 wherein said enteric contrast agent is administered to said subject by delivery through:
(a) a natural cavity selected from the mouth, vagina, bladder, rectum and urethra;
(b) a surgically created space selected from an ileal pouch, Hartmans pouch, and a neobladder;
(c) a space created by injury selected from a fistula, sinus tract, and abscess; or
(d) a medical device selected from a catheter, a tube, a reservoir, a pouch and a pump.

27. A kit comprising:
(a) a first vial or set of vials containing the enteric contrast medium of claim 1;
(b) a second vial containing a second contrast medium; and
(c) directions for formulating said enteric contrast medium with or without said second contrast medium.

28. The method of claim 19, comprising diagnosing said subject.

29. The method of claim 28, wherein said subject is diagnosed as having injury selected from a malignancy, inflammation, infection, and ischemia, and a combination thereof.

30. The method of claim 29, wherein said subject is evaluated for anatomical detail that involves the bowel or tissues adjacent to bowel.
